(19) United States
(12) Patent Application Publication
(10) Pub. No.: US 2022/0220565 A1
(43) Pub. Date: Jul. 14, 2022

(54) METHODS AND COMPOSITIONS FOR TREATING MELANOMA

(71) Applicants: INSERM (INSTITUT NATIONAL DE LA SANTÉ ET DE LA RECHERCHE MÉDICALE), Paris (FR); UNIVERSITE COTE D'AZUR, Nice (FR)

(72) Inventors: Marcel DECKERT, Nice Cedex 3 (FR); Sophie TARTARE - DECKERT, Nice Cedex 3 (FR); Moeez RATHORE, Austin, MN (US); Mélanie TICHET, Luasanne (CH)

(21) Appl. No.: 17/607,552

(22) PCT Filed: Apr. 29, 2020
(86) PCT No.: PCT/EP2020/061869

§ 371 Date: Oct. 29, 2021

(30) Foreign Application Priority Data
Apr. 30, 2019 (EP) 19305555.5

(51) Int. Cl. C12Q 1/6886; A61P 35/00; C12N 15/113; A61K 31/713; A61K 45/06

(52) U.S. Cl. CPC C12Q 1/6886; A61P 35/00; C12N 15/113; A61K 31/713; C12N 2320/31; C12Q 2600/112; C12Q 2600/118; C12Q 2600/158; C12N 2310/14; A61K 45/06

(57) ABSTRACT

The present invention relates to a method and composition for treating melanoma, aggressive/invasive melanoma, metastatic melanoma or melanoma resistant. More particularly, inventors have shown that high expression of PTX3 correlates with melanoma invasiveness and with a poorer survival rate in metastatic melanoma patients. PTX3 knockdown inhibited melanoma cell migration, invasion, lung metastasis, and NFκB signaling pathway. An addition of melanoma-derived or recombinant PTX3, or overexpression of PTX3 enhanced motility of low migratory cells. Finally, they found that TLR4 and MYD88 knockdown or targeting inhibited PTX3-induced melanoma cell migration, suggesting that PTX3 functions through a TLR4/NFκB-dependent pathway. Accordingly, the invention relates to a method for predicting the survival time of a subject suffering from melanoma, aggressive/invasive melanoma, metastatic melanoma or melanoma resistant by quantifying the expression level of PTX3 in a biological sample and to a method of treating melanoma, aggressive/invasive melanoma, metastatic melanoma or melanoma resistant by using the inhibitors of PTX3.

### FIELD OF THE INVENTION

The invention is in the field of oncology, more particularly the invention relates to method and compositions for treating melanoma, aggressive/invasive melanoma, metastatic melanoma or melanoma resistant.

### BACKGROUND OF THE INVENTION

Melanoma is the leading cause of skin cancer-related deaths, whose incidence has increased dramatically in the past 30 years. Melanoma arises from neural-crest-derived melanocytes found in the basal layer of the epidermis and responsible for melanin production and pigmentation. Early metastatic spread, intratumor heterogeneity and therapeutic resistance are hallmarks of melanoma. During the last years, much has been learned about the genetic and epigenetic driver events of tumor initiation, progression, and response to targeted therapies. Activating mutations of the BRAF proto-oncogene, which are the most prevalent mutations predict clinical efficacy to BRAF inhibitors such as vemurafenib in BRAF-mutated metastatic melanomas; and NRAS mutant tumors are known to exhibit sensitivity to MEK inhibition [1, 2]. Despite recent breakthroughs in melanoma treatments with the combination of targeted therapies using BRAF and MEK inhibitors and immunotherapies using checkpoint-blocking antibodies, resistance invariably develops and the prognosis remains dismal for most patients. Notably, current targeted therapies have even been shown to promote a mesenchymal-like invasive cell state and metastasis of resistant melanoma cells [3].

The acquisition of an invasive behavior is one of the key events in the progression to aggressive and deadly melanoma. Thus, identification of tumor cell intrinsic and extrinsic factors and characterization of the molecular mechanisms that drive this behavior are essential to our understanding of how melanoma metastasis develops, and to the development of novel therapeutic strategies. Emerging evidence support that progression to metastasis involves a series of reversible phenotypic changes in tumor cells. Under microenvironmental influences melanoma cells can undergo transcriptional reprogramming and switch between two different dominant states, either proliferative and poorly invasive or invasive and poorly proliferative, both being characterized by distinct gene expression signatures [3-6]. Levels and activity of microphthalmia-associated transcription factor (MITF), the melanocyte lineage-restricted transcription factor, are key determinants of the melanoma phenotype switch and tumor cell plasticity. The “proliferative” cellular state expresses higher levels of MITF than the “invasive” state. Importantly, the MITF^{low} invasive signature has been associated with intrinsic resistance to new drugs targeting mutant BRAF and MEK [3, 7, 8]. Finally, acquisition of migratory and invasive traits by melanoma cells also involved modulation of epithelial-mesenchymal transition (EMT)-inducing transcription factors expression including members of the SNAIL, TWIST and ZEB families [9-11].

Tumor cell-secreted factors play a key role in the process of malignant progression via their ability to regulate cell autonomous signaling and paracrine stroma communication. Drivers of melanoma invasion include such autocrine signals propagated by secreted factors that may thus represent potential candidate molecules for diagnosis and specific anti-invasive therapies.

Accordingly, there is a need to find new therapeutically tools to predict malignant progression of melanoma and treat resistant melanoma.

### SUMMARY OF THE INVENTION

The invention relates to a method for treating melanoma, aggressive/invasive melanoma, metastatic melanoma or melanoma resistant in a subject in need thereof comprising a step of administering said subject with a therapeutically effective amount of an inhibitor of PTX3. In particular, the present invention is defined by the claims.

### DETAILED DESCRIPTION OF THE INVENTION

Inventors have investigated the nature of the invasive secretome by using a comparative proteomic approach. Here, they have extended this analysis to show that PTX3, an acute phase inflammatory glycoprotein, is one such factor secreted by invasive melanoma to promote tumor cell invasiveness. Elevated PTX3 production was observed in the population of MITF^{low} invasive cells but not in a population of MITF^{high} differentiated melanoma cells. PTX3 was also found highly expressed in BRAF inhibitor-resistant melanoma cells displaying a mesenchymal invasive phenotype. Consistently, MITF knockdown induced PTX3 expression in MITF^{high} proliferative and poorly invasive cells. Importantly, high levels of PTX3 were found in tissues and blood of metastatic melanoma patients, and in samples from relapsed patients treated with the BRAF inhibitor. Mechanistically, autocrine production of PTX3 by melanoma cells triggered an IKK/NFκB signaling pathway that promotes migration, invasion, and expression of the pro-metastatic factor TWIST1. In contrast, addition of melanoma-derived or recombinant PTX3, or overexpression of PTX3 enhanced motility of low migratory cells. Finally, they found that TLR4 and MYD88 knockdown or targeting inhibited PTX3-induced melanoma cell migration, suggesting that PTX3 functions through a TLR4-dependent pathway. Their work reveals that tumor-derived PTX3 contributes to melanoma cell invasion via targetable inflammation-related pathways. In addition to providing new insights into the biology of melanoma invasive behavior, this study underscores the notion that secreted PTX3 represents a potential biomarker and therapeutic target in metastatic and/or refractory melanoma.

Method for Predicting the Survival Time of a Subject Suffering From Melanoma, Aggressive/Invasive Melanoma, Metastatic Melanoma or Melanoma Resistant

In a first aspect, the invention relates to a method for predicting the survival time of a subject suffering from melanoma, aggressive/invasive melanoma, metastatic melanoma or melanoma resistant comprising the steps of i) quantifying the expression level of PTX3 in a biological sample obtained from the subject; ii) comparing the expression level quantified at step i) with its predetermined reference value and iii) concluding that the subject will have a short survival time when the expression level of PTX3 is higher than its predetermined reference value or concluding that the subject will have a long survival time when the expression level of PTX3 is lower than its predetermined reference value.

In a particular embodiment, the invention relates to a method for predicting the survival time of a subject suffering from melanoma comprising the steps of i) quantifying the expression level of PTX3 in a biological sample obtained from the subject; ii) comparing the expression level quantified at step i) with its predetermined reference value and iii) concluding that the subject will have a short survival time when the expression level of PTX3 is higher than its predetermined reference value or concluding that the subject will have a long survival time when the expression level of PTX3 is lower than its predetermined reference value.

In a particular embodiment, the invention relates to a method for predicting the survival time of a subject suffering from an aggressive/invasive melanoma or metastatic melanoma comprising the steps of i) quantifying the expression level of PTX3 in a biological sample obtained from the subject; ii) comparing the expression level quantified at step i) with its predetermined reference value and iii) concluding that the subject will have a short survival time when the expression level of PTX3 is higher than its predetermined reference value or concluding that the subject will have a long survival time when the expression level of PTX3 is lower than its predetermined reference value.

Typically, an increased expression level of PTX3 in the biological sample obtained from the subject suffered from melanoma compared to the predetermined reference value indicates that the melanoma is aggressive/invasive or that the melanoma is metastatic.

In another embodiment, the invention relates to a method for predicting the survival time of a subject suffering from melanoma resistant comprising the steps of i) quantifying the expression level of PTX3 in a biological sample obtained from the subject; ii) comparing the expression level quantified at step i) with its predetermined reference value and iii) concluding that the subject will have a short survival time when the expression level of PTX3 is higher than its predetermined reference value or concluding that the subject will have a long survival time when the expression level of PTX3 is lower than its predetermined reference value.

The method is particularly suitable for predicting the duration of the overall survival (OS), progression-free survival (PFS) and/or the disease-free survival (DFS) of the cancer subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they have become cancer-free (achieved remission). DSF gives more specific information and is the number of people with a particular cancer who achieve remission. Also, progression-free survival (PFS) rates (the number of people who still have cancer, but their disease does not progress) include people who may have had some success with treatment, but the cancer has not disappeared completely. As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

As used herein, the term “melanoma” also known as malignant melanoma, refers to a type of cancer that develops from the pigment-containing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma, is a rare form of melanoma that occurs in the eye. In a particular embodiment, the melanoma is cutaneous melanoma. In a particular embodiment, the subject has or is susceptible to have an aggressive/invasive melanoma. In a particular embodiment, the subject has or is susceptible to have metastatic melanoma. In a particular embodiment, the subject has or is susceptible to have melanoma resistant.

As used herein, the terms “aggressive” and “invasive” are used herein interchangeably. When used herein to characterize a melanoma, they refer to the proclivity of a tumor for expanding beyond its boundaries into adjacent tissue. Invasive melanoma can be contrasted with organ-confined cancer wherein the tumor is confined to a particular organ or to a particular location in an organ. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

As used herein, the term “metastatic melanoma” refers to the spread of melanoma tumor cells from one organ or tissue to another location. The term also refers to tumor tissue that forms in a new location as a result of metastasis. A “metastatic cancer” is a cancer that spreads from its original, or primary, location, and may also be referred to as a “secondary cancer” or “secondary tumor”. Generally, metastatic tumors are named for the tissue of the primary tumor from which they originate.

As used herein, the term “melanoma resistant” refers to melanoma which does not respond to a treatment. The cancer may be resistant at the beginning of treatment or it may become resistant during treatment. The resistance to drug leads to rapid progression of metastatic of melanoma. The resistance of cancer for the medication is caused by mutations in the gene which are involved in the proliferation, divisions or differentiation of cells. In the context of the invention, the resistance of melanoma is caused by the mutations (single or double) in the following genes: BRAF, MEK or NRAS. The resistance can be also caused by a double-negative BRAF and NRAS mutation.

In a particular embodiment, the melanoma resistant is resistant to classical treatment.

In a particular embodiment, the melanoma resistant is resistant to radiation therapy, immunotherapy or chemotherapy.

In a particular embodiment, the melanoma is resistant to a treatment with the inhibitors of BRAF mutations. BRAF is a member of the Raf kinase family of serine/threonine-specific protein kinases. This protein plays a role in regulating the MAP kinase/ERKs signaling pathway, which affects cell division, differentiation, and secretion. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T598I, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E.

The inhibitors of BRAF mutations are well known in the art. In a particular embodiment, the melanoma is resistant to a treatment with vemurafenib. Vemurafenib also known as

PLX4032, RG7204 ou R05185426 and commercialized by Roche as Zelboraf. In a particular embodiment, the melanoma is resistant to a treatment with dacarbazine. Dacarbazine also known as imidazole carboxamide is commercialized as DTIC-Dome by Bayer. In a particular embodiment, the melanoma is resistant to a treatment with dabrafenib also known as tafinlar which is commercialized by Novartis.

In a further embodiment, the melanoma is resistant to a treatment with the inhibitors of MEK. MEK refers to Mitogen-activated protein kinase kinase, also known as MAP2K, MEK, MAPKK. It is a kinase enzyme which phosphorylates mitogen-activated protein kinase (MAPK). MEK is activated in melanoma. The inhibitors of MEK are well known in the art. In a particular embodiment, the melanoma is resistant to a treatment with trametinib also known as mekinist which is commercialized by GSK. In a particular embodiment, the melanoma is resistant to a treatment with cobimetinib also known as cotellic commercialized by Genentech. In a particular embodiment, the melanoma is resistant to a treatment with Binimetinib also knowns as MEK162, ARRY-162 is developed by Array Biopharma.

In a particular embodiment, the melanoma is resistant to a treatment with the inhibitors of NRAS. The NRAS gene is in the Ras family of oncogene and involved in regulating cell division. NRAS mutations in codons 12, 13, and 61 arise in 15-20% of all melanomas. The inhibitors of BRAF mutation or MEK are used to treat the melanoma with NRAS mutations. In a particular embodiment, the melanoma is resistant in which double-negative BRAF and NRAS mutant melanoma.

In a particular embodiment, the melanoma is resistant to a combined treatment. As used herein, the terms “combined treatment”, “combined therapy” or “therapy combination” refer to a treatment that uses more than one medication. The combined therapy may be dual therapy or bi-therapy. In the context of the invention, the melanoma is resistant to a combined treatment characterized by using an inhibitor of BRAF mutation and an inhibitor of MEK as described above. For example, the combined treatment may be a combination of vemurafenib and cotellic.

In a further embodiment, the melanoma is resistant to a treatment with an immune checkpoint inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e.g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3 -benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to -N-(3-bromo-4-fluorophenyl)-N′-hydroxy-4-{[2-(sulfamoylamino)-ethyl]amino}-1,2,5-oxadiazole-3 carboximidamide:

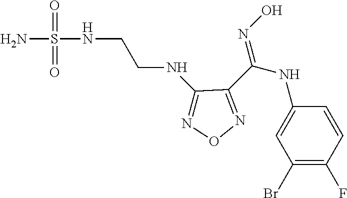 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

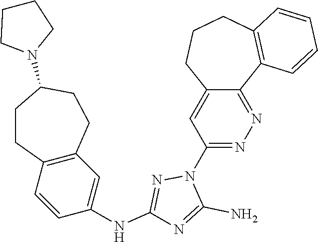 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the term “subject” denotes a mammal, such as a rodent, a feline, a canine, and a primate. Particularly, the subject according to the invention is a human. More particularly, the subject according to the invention has or is susceptible to have melanoma resistant. In particular embodiment, the subject has or is susceptible to have cutaneous melanoma. In a particular embodiment, the subject has or is susceptible to have metastatic melanoma.

As used herein, the term “PTX3” refers to Pentraxin-related protein PTX3 also known as TNF-inducible gene 14 protein (TSG-14). It is a protein that in humans is encoded by the PTX3 gene. The naturally occurring human PTX3 gene has a nucleotide sequence as shown in Genbank Accession number NM_002852 and the naturally occurring human PTX3 protein has an aminoacid sequence as shown in Genbank Accession number NP_002843. The murine nucleotide and amino acid sequences have also been described (Genbank Accession numbers NM_008987 and NP_033013). Pentraxins function as soluble pattern recognition molecules and one of the earliest and most important roles for these proteins is host defense primarily against pathogenic bacteria. They function as opsonins for pathogens through activation of the complement pathway and through binding to Fc gamma receptors. Pentraxins also recognize membrane phospholipids and nuclear components exposed on or released by damaged cells. More particularly, PTX3, activates complement, binds to FcyRIII, protects from some fungal infections and may play a role in wound healing.

As used herein, the term “expression level” refers to the expression level of PTX3. Typically, the expression level of the PTX3 gene may be determined by any technology known by a person skilled in the art. In particular, each gene expression level may be measured at the genomic and/or nucleic and/or protein level. In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined by using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids do not need to be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50 formamide, 5x or 6x SCC. SCC is a 0.15 M NaCl, 0.015 M Na-citrate). The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences. In a particular embodiment, the method of the invention comprises the steps of providing total RNAs extracted from a biological sample and subjecting the RNAs to amplification and hybridization to specific probes, more particularly by means of a quantitative or semi-quantitative RT-PCR. In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

As used herein, the term “biological sample” refers to any sample obtained from a subject, such as a serum sample, a plasma sample, a urine sample, a blood sample, a lymph sample, or a tissue biopsy. In a particular embodiment, biological sample for the determination of an expression level include samples such as a blood sample, a lymph sample, or a biopsy.

In a particular embodiment, the biological sample is a blood sample, more particularly, peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art.

In a particular embodiment, the biological sample is tissue sample.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of
a) providing a collection of tumor tissue samples from subject suffering from melanoma;
b) providing, for each tumor tissue sample provided at step a), information relating to the actual clinical outcome for the corresponding subject (i.e. the duration of the disease-free survival (DFS) and/or the overall survival (OS));
c) providing a serial of arbitrary quantification values;
d) quantifying the cell density for each tumor tissue sample contained in the collection provided at step a);
e) classifying said tumor tissue samples in two groups for one specific arbitrary quantification value provided at step c), respectively: (i) a first group comprising tumor tissue samples that exhibit a quantification value for level that is lower than the said arbitrary quantification value contained in the said serial of quantification values; (ii) a second group comprising tumor tissue samples that exhibit a quantification value for said level that is higher than the said arbitrary quantification value contained in the said serial of quantification values; whereby two groups of tumor tissue samples are obtained for the said specific quantification value, wherein the tumor tissue samples of each group are separately enumerated;
f) calculating the statistical significance between (i) the quantification value obtained at step e) and (ii) the actual clinical outcome of the subjects from which tumor tissue samples contained in the first and second groups defined at step f) derive;
g) reiterating steps f) and g) until every arbitrary quantification value provided at step d) is tested;
h) setting the said predetermined reference value as consisting of the arbitrary quantification value for which the highest statistical significance (most significant P-value obtained with a log-rank test, significance when P<0.05) has been calculated at step g).

For example the cell density has been assessed for 100 tumor tissue samples of 100 subjects. The 100 samples are ranked according to the cell density. Sample 1 h as the highest density and sample 100 h as the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off” value as described above. For example, according to this specific embodiment of a “cut-off” value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

In a particular embodiment, the method according to the invention further comprises a step of classification of subject by an algorithm and determining whether a subject will have a long survival time.

Typically, the method of the present invention comprises a) quantifying the level of the PTX3 in the biological sample; b) implementing a classification algorithm on data comprising the quantified of PTX3 levels so as to obtain an algorithm output; c) determining the probability that the subject have a long survival time from the algorithm output of step b).

In some embodiments, the method according to the invention wherein the algorithm is selected from Linear Discriminant Analysis (LDA), Topological Data Analysis (TDA), Neural Networks, Support Vector Machine (SVM) algorithm and Random Forests algorithm (RF).selected from Linear Discriminant Analysis (LDA), Topological Data Analysis (TDA), Neural Networks, Support Vector Machine (SVM) algorithm and Random Forests algorithm (RF).

In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase.In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

Method for Treating Melanoma, Aggressive/Invasive Melanoma, Metastatic Melanoma or Melanoma Resistant

Inventors have shown that an inhibition of PTX3 by siRNAs and shRNA reduced the invasive migration of melanoma cells and melanoma resistant cells.

Accordingly, in a second aspect, the invention relates to a method for treating melanoma, aggressive/invasive melanoma, metastatic melanoma or melanoma resistant in a subject in need thereof comprising a step of administering said subject with a therapeutically effective amount of an inhibitor of PTX3.

In a particular embodiment, the subject is identified as having a short survival time by performing the method as described above.

In a particular embodiment, the subject shows an aberrant and increased expression of PTX3.

In a particular embodiment, the invention relates to a method for treating an aggressive/invasive melanoma or metastatic melanoma in a subject in need thereof comprising a step of administering said subject with a therapeutically effective amount of an inhibitor of PTX3.

In another embodiment, the invention relates to a method for treating melanoma resistant in a subject in need thereof comprising a step of administering said subject with a therapeutically effective amount of an inhibitor of PTX3.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The term “inhibitor of PTX3” refers to a natural or synthetic compound that has a biological effect to inhibit the activity or the expression of PTX3. More particularly, inhibition of PTX3 activity or expression reduces NF-kκB activity. More particularly, such compound by inhibiting PTX3 activity or expression reduces the invasive migration and motility of melanoma resistant cells. Such inhibitor is able to inhibit the migration, aggressively and invasiveness of melanoma in a subject. As used herein, the term “inhibit” means to prevent something from happening, to delay occurrence of something happening, and/or to reduce the extent or likelihood of something happening. Thus, the terms “inhibiting metastasis”, “inhibiting metastases” and “inhibiting the formation of metastases”, which are used herein interchangeably, are intended to encompass preventing, delaying, and/or reducing the likelihood of occurrence of metastases as well as reducing the number, growth rate, size, etc. . . . of metastases.

In a particular embodiment, the inhibitor of PTX3 is a peptide, peptidomimetic, small organic molecule, antibody, aptamers, siRNA or antisense oligonucleotide.

The term “peptidomimetic” refers to a small protein-like chain designed to mimic a peptide. In a particular embodiment, the inhibitor of PTX3 is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the inhibitor of PTX3 is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In some embodiments, the inhibitor of PTX3 expression is a short hairpin RNA (shRNA), a small interfering RNA (siRNA) or an antisense oligonucleotide which inhibits the expression of PTX3. In a particular embodiment, the inhibitor of PTX3 expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PTX3 expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156 : 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the inhibitor of PTX3 is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001 ; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the inhibitor is an intrabody having specificity for PTX3. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

As used herein, the term “subject” denotes a mammal, such as a rodent, a feline, a canine, and a primate. Particularly, the subject according to the invention is a human.

In one embodiment, the subject according to the invention shows an aberrant and increased expression of PTX3.

More particularly, the subject according to the invention has or susceptible to have melanoma resistant. In a particular embodiment, the subject has or susceptible to have melanoma resistant to at least one of the treatments as described above. The subject having a melanoma resistant is identified by standard criteria. The standard criteria for resistance, for example, are Response Evaluation Criteria In Solid Tumors (RECIST) criteria, published by an international consortium including NCI.

As used herein the terms “administering” or “administration” refer to the act of injecting or otherwise physically delivering a substance as it exists outside the body (e.g., an inhibitor of PTX3) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of inhibitor of PTX3 for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Combined Preparation

In a third aspect, the invention relates to i) a PTX3 inhibitor and ii) a classical treatment as a combined preparation for use in the treatment of melanoma, aggressive/invasive melanoma, metastatic melanoma, melanoma resistant.

In a particular embodiment, i) a PTX3 inhibitor for use according to the invention, and ii) a classical treatment as a combined preparation for use in the treatment of aggressive/invasive melanoma.

In a particular embodiment, i) a PTX3 inhibitor for use according to the invention, and ii) a classical treatment as a combined preparation for use in the treatment of metastatic melanoma.

In a particular embodiment, i) a PTX3 inhibitor for use according to the invention, and ii) a classical treatment as a combined preparation for simultaneous, separate or sequential use in the treatment of melanoma, aggressive/invasive melanoma, metastatic melanoma or melanoma resistant.

In a particular embodiment, i) a PTX3 inhibitor for use according to the invention, and ii) a classical treatment as a combined preparation for simultaneous, separate or sequential use in the treatment of melanoma.

In a particular embodiment, i) a PTX3 inhibitor for use according to the invention, and ii) a classical treatment as a combined preparation for simultaneous, separate or sequential use in the treatment of aggressive/invasive melanoma.

In a particular embodiment, i) a PTX3 inhibitor for use according to the invention, and ii) a classical treatment as a combined preparation for simultaneous, separate or sequential use in the treatment of metastatic melanoma.

In a particular embodiment, i) a PTX3 inhibitor for use according to the invention, and ii) a classical treatment as a combined preparation for simultaneous, separate or sequential use in the treatment of melanoma resistant.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The PTX3 inhibitor can be used alone as a single inhibitor or in combination with other a classical treatment. When several inhibitors are used, a mixture of inhibitors is obtained. In the case of multi-therapy (for example, bi-, tri- or quadritherapy), at least on other inhibitor can accompany the PTX3 inhibitor.

As used herein, the term “classical treatment” refers to treatments well known in the art and used to treat melanoma. In the context of the invention, the classical treatment refers to radiation therapy, immunotherapy or chemotherapy.

In a particular embodiment, the invention relates i) a PTX3 inhibitor and ii) a chemotherapy used as a combined preparation for use in the treatment of melanoma, aggressive/invasive melanoma, metastatic melanoma or melanoma resistant.

In a particular embodiment, i) a PTX3 inhibitor and ii) chemotherapy as a combined preparation according to the invention for simultaneous, separate or sequential use in the use in the treatment of melanoma, aggressive/invasive melanoma, metastatic melanoma or melanoma resistant.

In a particular embodiment, i) a PTX3 inhibitor and ii) chemotherapy as a combined preparation according to the invention for simultaneous, separate or sequential use in the use in the treatment of aggressive/invasive melanoma.

In a particular embodiment, i) a PTX3 inhibitor and ii) chemotherapy as a combined preparation according to the invention for simultaneous, separate or sequential use in the use in the treatment of metastatic melanoma.

In a particular embodiment, i) a PTX3 inhibitor and ii) chemotherapy as a combined preparation according to the invention for simultaneous, separate or sequential use in the use in the treatment of melanoma resistant.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the invention relates i) a PTX3 inhibitor and ii) a radiotherapy used as a combined preparation for use in the treatment of melanoma resistant.

In a particular embodiment, i) a PTX3 inhibitor and ii) radiotherapy as a combined preparation according to the invention for simultaneous, separate or sequential use in the use in the treatment of melanoma, aggressive/invasive melanoma, metastatic melanoma or melanoma resistant.

In a particular embodiment, i) a PTX3 inhibitor and ii) radiotherapy as a combined preparation according to the invention for simultaneous, separate or sequential use in the use in the treatment of metastatic melanoma.

In a particular embodiment, i) a PTX3 inhibitor and ii) radiotherapy as a combined preparation according to the invention for simultaneous, separate or sequential use in the use in the treatment of melanoma resistant.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates i) a PTX3 inhibitor and ii) an immune checkpoint inhibitor used as a combined preparation for the treatment melanoma, aggressive/invasive melanoma, metastatic melanoma or melanoma resistant.

In a particular embodiment, i) a PTX3 inhibitor and ii) an immune checkpoint inhibitor as a combined preparation according to the invention for simultaneous, separate or sequential use in the treatment of melanoma, aggressive/invasive melanoma, metastatic melanoma or melanoma resistant.

In a particular embodiment, i) a PTX3 inhibitor and ii) an immune checkpoint inhibitor as a combined preparation according to the invention for simultaneous, separate or sequential use in the treatment of melanoma.

In a particular embodiment, i) a PTX3 inhibitor and ii) an immune checkpoint inhibitor as a combined preparation according to the invention for simultaneous, separate or sequential use in the treatment of aggressive/invasive melanoma.

In a particular embodiment, i) a PTX3 inhibitor and ii) an immune checkpoint inhibitor as a combined preparation according to the invention for simultaneous, separate or sequential use in the treatment of metastatic melanoma.

In a particular embodiment, i) a PTX3 inhibitor and ii) an immune checkpoint inhibitor as a combined preparation according to the invention for simultaneous, separate or sequential use in the treatment of melanoma resistant.

In a particular embodiment, the PTX3 inhibitor and an immune checkpoint inhibitor as a combined preparation according to the invention, wherein the immune checkpoint inhibitor is selected from the group consisting of but not limited to: Nivolumab (Opdivo®, BMS), Pembrolizumab (also called Lambrolizumab, KEYTRUDA® or MK-3475, MERCK). Atezolizumab (MPDL3280A, Genentech/Roche), Durvalumab (AZD9291, AstraZeneca), Avelumab (also known as MSB0010718C, Merck) and BMS-936559 (BMS). In particular embodiment, immune checkpoint inhibitor is described above.

In a particular embodiment, i) a PTX3 inhibitor and ii) a BRAF inhibitor as a combined preparation for use in the treatment melanoma, an aggressive/invasive melanoma, metastatic melanoma or melanoma resistant.

In a particular embodiment, i) a PTX3 inhibitor and ii) a BRAF inhibitor as a combined preparation according to the invention for simultaneous, separate or sequential use in the treatment of melanoma, aggressive/invasive melanoma, metastatic melanoma or melanoma resistant.

In a particular embodiment, i) a PTX3 inhibitor and ii) a BRAF inhibitor as a combined preparation according to the invention for simultaneous, separate or sequential use in the treatment of melanoma.

In a particular embodiment, i) a PTX3 inhibitor and ii) a BRAF inhibitor as a combined preparation according to the invention for simultaneous, separate or sequential use in the treatment of aggressive/invasive melanoma.

In a particular embodiment, i) a PTX3 inhibitor and ii) a BRAF inhibitor as a combined preparation according to the invention for simultaneous, separate or sequential use in the treatment of metastatic melanoma.

In a particular embodiment, i) a PTX3 inhibitor and ii) a BRAF inhibitor as a combined preparation according to the invention for simultaneous, separate or sequential use in the treatment of melanoma resistant.

In a particular embodiment, the PTX3 inhibitor and BRAF inhibitor as a combined preparation according to the invention, wherein the BRAF inhibitor is selected from the group consisting of but not limited to: Vemurafenib, Dacarbazine or Dabrafenib.

In a particular embodiment, i) a PTX3 inhibitor and ii) a MEK inhibitor as a combined preparation for use in the treatment of melanoma, an aggressive/invasive melanoma, metastatic melanoma or melanoma resistant.

In a particular embodiment, i) a PTX3 inhibitor and ii) a MEK inhibitor as a combined preparation according to the invention for simultaneous, separate or sequential use in the treatment of melanoma, aggressive/invasive melanoma, metastatic melanoma or melanoma resistant.

In a particular embodiment, i) a PTX3 inhibitor and ii) a MEK inhibitor as a combined preparation according to the invention for simultaneous, separate or sequential use in the treatment of aggressive/invasive melanoma.

In a particular embodiment, i) a PTX3 inhibitor and ii) a MEK inhibitor as a combined preparation according to the invention for simultaneous, separate or sequential use in the treatment of metastatic melanoma.

In a particular embodiment, i) a PTX3 inhibitor and ii) a MEK inhibitor as a combined preparation according to the invention for simultaneous, separate or sequential use in the treatment of melanoma resistant.

In a particular embodiment, the PTX3 inhibitor and MEK inhibitor as a combined preparation according to the invention, wherein the MEK inhibitor is selected from the group consisting of but not limited to: Trametinib, Cobimetinib or Binimetinib.

### Pharmaceutical Composition

The inhibitor of PTX3 for use according to the invention alone and/or combined with classical treatment as described above may be combined with pharmaceutically acceptable excipients, and optionally sustained-release matrices, such as biodegradable polymers, to form pharmaceutical compositions.

Accordingly, in a fourth aspect, the invention relates to a pharmaceutical composition comprising a PTX3 inhibitor for use in the treatment of melanoma, aggressive/invasive melanoma, metastatic melanoma or melanoma resistant.

In a particular embodiment, the pharmaceutical composition according the invention, wherein the PTX3 inhibitor is siRNA.

In a particular embodiment, the pharmaceutical composition according the invention, wherein the PTX3 inhibitor is a small molecule.

In a particular embodiment, the pharmaceutical composition according the invention comprising i) a PTX3 inhibitor and ii) a classical treatment.

The inhibitors of PTX3 and the combined preparation as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Kit

In another aspect, the invention relates to a kit suitable to predict the survival time of a subject suffering or susceptible to suffer from melanoma, an aggressive/invasive melanoma, metastatic melanoma or melanoma resistant.

Accordingly, the invention relates to a kit for use in the method for predicting the survival time of a subject having or susceptible to have melanoma, an aggressive/invasive melanoma, metastatic melanoma or melanoma resistant said kit comprising a reagent that specifically reacts with PTX3 mRNA or protein and instructions to perform the predicting method of the survival time according to the method as described above.

The kit for the use according to the invention, wherein the reagent that specifically reacts with PTX3 mRNA or protein is selected from the group consisting of oligonucleotide probes that specifically hybridize to PTX3 mRNA transcripts, oligonucleotide primers that specifically amplify PTX3 mRNA transcripts, antibodies that specifically recognize/bind the PTX3 protein, and PTX3-binding peptides that specifically bind to the PTX3 protein.

### Method of Screening

In a another aspect, the present invention relates to a method of screening a drug suitable for the treatment of melanoma, aggressive/invasive melanoma, metastatic melanoma or melanoma resistant comprising i) providing a test compound and ii) determining the ability of said test compound to inhibit the activity of PTX3.

Any biological assay well known in the art could be suitable for determining the ability of the test compound to inhibit the activity of PTX3. In some embodiments, the assay first comprises determining the ability of the test compound to bind to PTX3. In some embodiments, a population of cells is then contacted and activated so as to determine the ability of the test compound to inhibit the activity of PTX3. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of PTX3, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: PTX3 expression is associated with an invasive MITFlow melanoma cell state. (a) ELISA analyses of PTX3 secretion in CM of different melanoma cell lines and patient short-term cultures. Equal amounts of CM from SBc12, WM35, WM793, 501Mel, MeWo, SKMe128, 1205Lu, A375, and melanoma patient cells (Pt1, Pt2) were subjected to PTX3 ELISA. Data represent the mean concentration of PTX3 in CM±s.e.m of duplicate determinations. (b) Equal amounts of CM from melanoma cell lines were immunoblotted with PTX3 antibody. Ponceau staining was used as a loading control for secreted proteins. (c) Box and whisker plots (10th to 90th percentile) show MITF and PTX3 expression across proliferative versus invasive cell states of melanoma cultures within the Mannheim, Philadelphia, and Zurich cohorts (GSE4843, GSE4841 and GSE4840 data sets). P values were calculated by two-tailed Mann Whitney test. ***P<0.0001; **P<0.01. (d) Spearman's correlation analysis of the MITF and PTX3 mRNA expression across the Mannheim melanoma cohort (GSE4843). (e) PTX3 mRNA expression in melanoma cell lines classified based on high (MITF high) or low (MITF low) levels of MITF expression. Box and whisker plots (10th to 90th percentile) show normalized PTX3 expression values mined from publicly available data sets (GSE62526 and GSE61544). P values were calculated by two-tailed Mann Whitney test with Gaussian approximation. ***P=0.0002; *P<0.05. (f) PTX3 and TYRP1 gene mRNA in melanoma cells depleted (shMITF) or not (shSCR) of MITF. Box plots depict PTX3 expression values extracted from a GEO data set (GSE50686). (g) 501Mel cells were transfected with siCTRL or siMITF for 3 days. Total cell lysates and CM were analyzed by immunoblotting with antibodies against MITF, p27Kip1, SPARC, PTX3 and ERK2 as a loading control.

FIG. 2: Expression of PTX3 in malignant melanoma. (a) Box and Whisker plots show PTX3 mRNA expression in metastatic vs benign skin lesions (nevi) (GSE3189). (b) Kaplan-Meier overall survival and disease-free survival curves for melanoma patients with or without PTX3 alterations (PTX3 amplifications and mRNA upregulation). Data were retrieved from the skin melanoma TCGA database using cBioPortal (cbioportal.org). Data were restricted to AMP EXP>=2 to exclude patients with PTX3 deletion. Median months survival: 28 vs 94.9 (p=9.872e-3, logrank test). Median months Disease-free: 25.5 vs 51.4 (p=0.0151, logrank test). (c) Quantification of PTX3 expression in stage III (6 cases) and stage IV (10 cases) melanoma tissue arrays from d and Supplementary FIG. 2. Serum analysis for PTX3 from metastatic melanoma (MM) patients (n=16) compared to healthy donors (n=10). Error bars represent mean s.e.m. ***P<0.0005, Mann-Whitney's test.

FIG. 3: Increased PTX3 expression in BRAF inhibitor mesenchymal resistant melanoma. (a) Immunoblot analyses of PTX3 expression in isogenic pairs of sensitive (M229S, M238S) and vemurafenib-resistant melanoma cells (M229R, M238R). Total cell lysates were immunoblotted with antibodies against PTX3, MITF, PDGFR-β, Fibronectin and HSP90 as a loading control. (b) ELISA analyses of PTX3 secretion in CM derived from the above sensitive and resistant melanoma cells. PTX3 levels in CM were normalized against the amount of cellular proteins in each condition. Data represent the mean concentration of PTX3 in CM ±s.e.m of duplicate determinations. (c) PTX3 gene expression levels in data sets (GSE5053) derived from three paired tumor biopsies of melanoma patients before (Pre) and after (Post) development of resistance to BRAF inhibitors. (d) CM was prepared from melanoma cell cultures derived from one patient before (Pre) and after (Post) development of drug resistance and immunoblotted with PTX3 and Fibronectin antibodies. Ponceau staining was used as a loading control for secreted proteins.

FIG. 4: Melanoma-derived PTX3 promotes tumor cell migration. (a) Metastatic (A375, 1205Lu), resistant (M229R, M238R) and patient-derived (Pt1) melanoma cells were transfected with siCTRL or two different siPTX3. After 3 days, serum-stimulated motility assays were performed for 4 h using Transwell inserts. Bar graphs show mean number of migrated cells±s.e.m (n=3 independent experiments). Lower panel: CMs from transfected cells were immunoblotted with anti-PTX3. (b) Migration assay for melanoma cells (501Mel, SKMel28) stably expressing PTX3 was performed using Transwell inserts for 16 h. Bar graphs show mean±s.e.m., n=3 independent experiments. PTX3 Immunoblots are shown in lower panel. (c) Melanoma cells (A375, 1205Lu) were transfected with siCTRL or siPTX3. After 3 days, serum-stimulated motility assays were performed in the presence or not of rhPTX3 (100 ng.ml-1). Bar graphs represent mean±s.e.m., n=3 independent experiments. (d) Confluent 501Mel cells were scratched and CMs from 1205Lu depleted or not for PTX3 were added. Cells were photographed under a phase contrast microscope after 48 h (upper panels) and migrating cells in scratch area were quantified (lower panels). Bar graphs represent means±s.e.m. (n=3 independent experiments). (e) Motility of 501Mel cells was assayed by scratch assays in the presence or not of 1205Lu CMs cleared from PTX3 by immunoprecipitation (IP). Upper panel: PTX3 immunoblot of the cleared CMs. Bar graphs show mean±s.e.m., n=2 independent experiments. (f) Confluent SKMel28 cell monolayers were wounded and rhPTX3 were added. Migrated cells in scratch area were photographed and quantified. Bar graphs represent mean±s.e.m (n=3 independent experiments performed in duplicate). **p<0.01; ***p<0.001 as determined by Student's t-test.

FIG. 5: PTX3 increases melanoma invasiveness in vitro. (a) Matrigel invasion assay was performed using Transwell inserts for siCTRL and siPTX3 transfected cells (1205Lu, A375, M229R) obtained from transfection performed for migration assays (FIG. 4a). Cells were left to migrate for 6 h and counted. Bar graphs show mean number of invaded cells±s.e.m (n=3 independent experiments). (b) 1205Lu cells were transfected with siCTRL or siPTX3 as described above. After 3 days, serum-stimulated matrigel invasion assays were performed in the presence or not of rhPTX3 (100ng.ml-1). Images of one representative insert out of three are shown (left). Bar graphs represent mean±s.e.m., n=3 independent experiments (right). (c) Matrigel invasion assay was performed as above for SKMel28 cells stably expressing PTX3 or not (FIG. 4b). Cells were left to migrate for 6 h and counted. Bar graphs show mean number s.e.m (n=3 independent experiments). (d) A375 cells were transfected with siPTX3 or siCTRL (50 nM). After 48 h, spheroids were implanted into collagen gels and incubated in growth medium for 3 days. Tumor cell outgrowth from spheroid edge was visualized by phase contrast microscopy and quantified by Image J. Scale bar represents 200 μM for all panels. Bar graphs show mean±s.e.m., n=2 independent experiments. ***P<0.001; **P<0.01. (e) Transendothelial migration assay was performed on monolayers of primary endothelial cells (HUVECs) grown on Transwell inserts. CMFDA-labeled 1205Lu cells transfected with siCTRL or siPTX3 were then added to the inserts. PTX3 knockdown cells were rescued by adding rhPTX3 (100 ng.ml-1). Cells were left to migrate for 6 h and cell tracker green positive cells were counted by fluorescent microscopy. Bar graphs show mean±s.e.m (n=2 independent experiments).

FIG. 6: PTX3 promotes melanoma invasiveness in experimental lung extravasation assay. (a) Immunoblot analysis of PTX3 expression on 1205Lu-Luc+ control (shCTRL) and stable PTX3 knockdown (shPTX3-5) cells (see Supplementary FIG. 7 for details). HSP60 was used as a loading control. (b) shCTRL and shPTX3-5 1205Lu-Luc+ were injected by tail vein into nude mice (n=5 mice per group) and lung metastasic progression was monitored and quantified using a photon imager. Representative images of photon fluxes produced by bioluminescent melanoma cells from day 0 to day 15 are shown (left panels, n=2) and normalized photon flux (BLI, right graph, n=5) of lungs from control and PTX3-depleted cells. (c) Quantification of lung metastatic foci by ex vivo BLI at the endpoint of the experimental lung extravasation assay performed in b. Representative ex vivo BLI imaging of lung mets (left) and quantification of lung metastatic foci per lung (right graph, n=5). (d) Quantification of lung colonization by 501Mel cells stably expressing PTX3 or not. Cell tracker-labelled melanoma cells that were present within the lungs were counted at the indicated time post injection. Bar graphs show mean±s.e.m. (n=6 randomly chosen fields) in left panel. Scale bar, 60 μm. **P<0.01, *P<0.05.

FIG. 7: TLR4/MYD88 expression is required for PTX3-induced melanoma cell migration. (a) Serum-stimulated migration was performed on 1205Lu cells transfected with siCTRL or 2 different siTLR4. Immunoblot analysis shows TLR4 depletion. HSP60, loading control. Bar graphs represent mean of migrated cells±s.e.m., n=3 independent experiments. (b) 1205Lu cells were transfected with siPTX3 or siTLR4. After 3 days, cells were stimulated or not with rhPTX3 for 4 h and serum-stimulated motility assays were performed. Bar graphs show migration mean±s.e.m., n=2 independent experiments. (c) Serum-stimulated migration on MYD88-depleted 1205Lu cells. Bar graphs show migration mean±s.e.m., n=2 independent experiments. MYD88 levels were controlled by immunoblot. (d) 1205Lu cells were treated with IKK inhibitor BMS-345541 and PTX3 expression was assessed by immunoblot. ***P<0.001, **P<0.01. (e) Schematic model of PTX3 autocrine action on melanoma cell migration and invasion.

### EXAMPLE

Material & Methods

Cells, Reagents and Plasmids

A375, 1205Lu and SKMe128 melanoma cell lines were purchased from ATCC (VA, USA). 1205Lu cells that were engineered to express a luciferase reporter (1205Lu-Luc+cells) were described before [12]. Other melanoma cell lines and patient melanoma cells were obtained as described before [50]. Melanoma cells were cultured in Dulbecco's modified Eagle Medium (DMEM) plus 7% Fetal Bovine Serum (FBS) (Thermo Fisher Scientific, MA, USA). Conditioned media (CM) from melanoma cells were prepared as previously described [12].

Human umbilical vascular endothelial cells (HUVECs) were cultured in complete EGM2 Bullet Kit medium supplemented with 2% FBS and full supplements (Lonza, Switzerland). All other culture reagents and Cell Tracker dyes were from Thermo Fischer Scientific. The source for other reagents was as follow: recombinant human PTX3 (Bio-Techne, MN, USA), MYD88 Inhibitor peptide (NOVUS Biologicals, CO, USA), IKK inhibitor BMS345541 (Selleckchem, TX, USA), chemicals (Sigma-Aldrich, MO, USA). The PTX3 cDNA was amplified by PCR from 1205Lu cells using the primers: Fwd:
ACTCGAGCCACCATGCATCTCCTTGCGATTCTG; (SEQ ID NO: 2) Rev: TAGGATCCTTATGAAACATACTGAGCTCCT (SEQ ID NO: 3) and subcloned in the pIRES2-EGFP vector (Takara, Japan) using BamHI/Xhol restriction sites to generate the pPTX3-IRES-EGFP vector. The PTX3-IRES-EGFP cassette was subsequently subcloned into the pLPCX retroviral vector using XhoI/NotI restriction sites to generate the pLPCX-PTX3-pIRES2-EGFP vector. 501Mel-CTRL and 501Mel-PTX3 cells were obtained following pIRES2-EGFP and pPTX3-IRES-EGFP transfection, respectively and geneticin selection. SKMel28-CTRL and SKMel28-PTX3 were generated by retroviral transduction with pLPCX and pLPCX-PTX3-IRES2-EGFP, respectively and puromycin selection. The NF-κB luciferase reporter vector was from BPS Bioscience (CA, USA).

RNAi Studies

Negative control, PTX3 siRNA duplexes were designed by Thermo Fisher Scientific. MITF (sc-35934), MYD88 (sc-35986) and TWIST1 (sc-38604) siRNAs were from Santa Cruz Biotechnology (TX, USA). IKKα, IKKβ, and TLR4 siRNAs were from Dharmacon (CO, USA). Transfection of siRNA was carried out using Lipofectamine RNAiMAX (Thermo Fisher Scientific), at a final concentration of 25 or 50 nM. Unless stated otherwise, cells were assayed 3 days post transfection. Lentiviral vectors for stable knockdown of PTX3 (NM_002852) in melanoma cells were TRC1.5-pLKO.1-puro vectors containing a s (shPTX3-1 to 5) (Genomic center, University of Minnesota, USA). Lentiviral, packaging (psPAX2) and enveloppe (pMD2.0G) vectors were transfected into HEK293T cells and virus-containing supernatant fractions were harvested after 72 h. The supernatant fractions were filtered and used to infect bioluminescent 1205Lu cells (1205Lu-Luc+) that were described before [12]. Transduced cells were selected with 2 μg/ml puromycin for 3 weeks. The efficiency of PTX3 knockdown was assessed by immunoblotting and analysis of in vitro cell migration using modified Boyden chambers. The PTX3 knockdown cells that were selected for in vivo studies were transduced by lentivirus harbouring the shPTX3-5 sequence (TRCN0000149744, 5′-CCGGGAGGAGCTCAGTATGTTTCATCTCGAGATGAAACATACTGAGCTCCTCTTT TTTG-3′) (SEQ ID NO: 1).

Immunohistochemistry and Immunoblot Analysis

Immunohistochemistry and immunoblot analysis were performed as described before [12]. IHC analysis was performed on melanoma tissue microarrays (US Biomax, MD, USA) using PTX3 antibody (HPA069320, Sigma-Aldrich; 1:50). For immunoblotting, the following antibodies were used at 1:1,000: SPARC (Bio-Techne), PTX3 (Enzo Life Sciences, Switzerland), HSP90, HSP60, TLR4, Fibronectin, ERK2 (Santa Cruz Biotechnology), MITF (Thermo Fisher Scientific), TWIST1 (Abcam, UK), SNAIL, phospho-IKKα/β (Ser176/180), NF-κB p65, phospho-NF-κB p65 (Ser536), I□B□ (L35A5), I□B□ (Ser32/36), IKKα, IKKβ, MYD88, phospho-AKT (Ser473), phospho-ERK1/2 (Thr202/Tyr204) (Cell Signaling Technology, MA, USA).

Migration, Invasion and In Vitro Scratch Assays

Serum-stimulated chemotaxis, invasion and transendothelial migration were monitored using modified Boyden chambers (8 μm pores, Sigma) as described before [12]. For invasion assays, the upper side of the filter was coated with 0.5 mg.ml-1 matrigel (Corning, N.Y., USA). Migrated cells were stained with crystal violet and counted (four fields randomly per well). For transendothelial migration, 105 HUVECs were grown in the upper chamber of gelatin-coated

Transwell inserts and treated with human TNFa (10 ng.ml-1) for 16 h. Melanoma cells were allowed to migrate for 6 h or 16 h and migrated cells were visualized and quantified as before [12]. Pictures of five random fields were captured for quantification using NIH ImageJ analysis software. For scratch assays, melanoma cells were grown approximately 80% confluence in 6-well plates. Wounds were generated using a sterile 200 μl pipette tip across each well. Following treatment with rhPTX3 or CM, pictures of four random fields were captured using an imaging station and the number of migrated cells in wounded area was counted with NIH ImageJ analysis software.

Spheroid Invasion Assay and Zymography

Melanoma three-dimensional (3D) spheroids were generated as described [51]. Pictures of migrating cells were taken at day 0 and day 3. Relative invasion was determined using the ImageJ software as the ratio between migration area at day 3 and migration area at day 0. CM from melanoma cells were analyzed by collagen zymography as previously described [52].

Promoter Reporter Assay

Melanoma cells were transfected with NF-κB-luciferase and β-Galactosidase reporter plasmids. 48 h after transfection, cells were lysed and luciferase and β-galactosidase activities were measured as described before [53]. Relative NF-κB promoter activity was calculated following normalization against β-galactosidase activity.

In Vivo Experiments and Immunostaining

All mouse experiments were carried out in accordance with the Institutional Animal Care and local ethics committees. For experimental lung metastasis studies, 5-week-old female nude mice (Harlan, IN, USA) were intravenously injected with 1205Lu Luc+cells (1×106) that were transduced with a non targeting shRNA lentivirus (shCTRL) or a PTX3 targeting shRNA lentivirus (shPTX3-5). Images were acquired using a Photon Imager (Biospace Lab, France) on mice injected i.p with 50mg.kg-1 D-luciferin (PerkinElmer, MA, USA). Lung metastasis was monitored and quantified using BLI [12]. In a second set of experiments, short-term lung colonization assays were performed with Cell Tracker Orange-stained 501Mel cells stably expressing PTX3 or not. Cells were intravenously injected in 5-week-old female nude mice that were sacrificed either 30 min or 24 h later. Lungs were perfused with PBS, fixed in 4% paraformaldehyde, OCT-embedded, cryosectioned, immunostained and imaged using a confocal microscope as described [12].

ELISA Assay

The levels of PTX3 in CM of melanoma cell lines or in human serum samples were measured using the human PTX3 Quantikine ELISA kit (Bio-Techne) as per the manufacturer's protocol. Serum samples were obtained from consenting metastatic melanoma patients through the Dermatology Department of Nice University Hospital (Nice, France) or from healthy volunteers as controls. Results are from two independent experiments performed in triplicate.

Analysis of Gene Expression From Human Databases

Publicly available gene expression data sets of human melanoma samples were used to analyze PTX3 levels in melanoma progression (GSE3189). From GEO database, we also examined the Mannheim (GSE4843), Philadelphia (GSE4841), and Zurich (GSE4840) cohorts. Proliferative and invasive melanoma subgroups were defined according to literature mined gene signatures [4]. Gene expression of PTX3 was also examined in data sets derived from patient tumour biopsies before and after development of drug resistance to Vemurafenib (GEO accession number GSE50535) [54]. Normalized data were analyzed using GraphPad Prism (GraphPad software, CA, USA). Survival data from the skin melanoma TCGA database were retrieved using cBioPortal (cbioportal.org). Gene Set Enrichment Analysis (GSEA) was performed as described before [55].

Statistical Analysis

Unless otherwise stated, all the experiments were repeated at least three times and representative data/images are shown. Statistical data analysis was performed using GraphPad Prism software. Unpaired two-tailed Mann-Whitney tests or two-way ANOVA tests with Bonferroni post-tests were used for statistical comparisons. Error bars are mean±s.e.m.

Results

PTX3 expression is Associated With an Invasive Melanoma Cell State

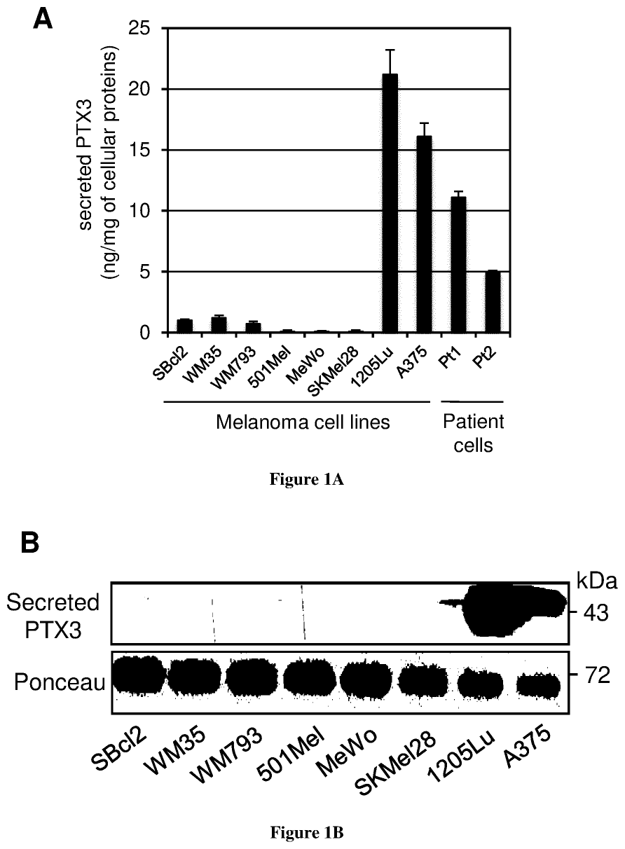

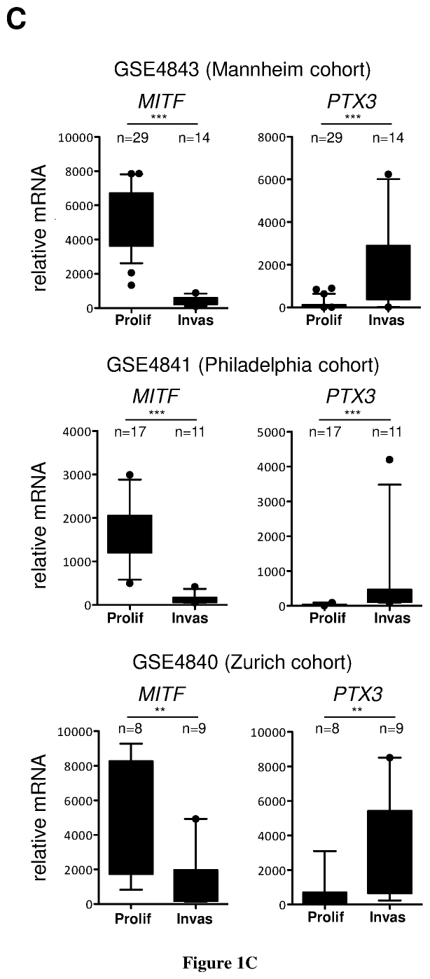

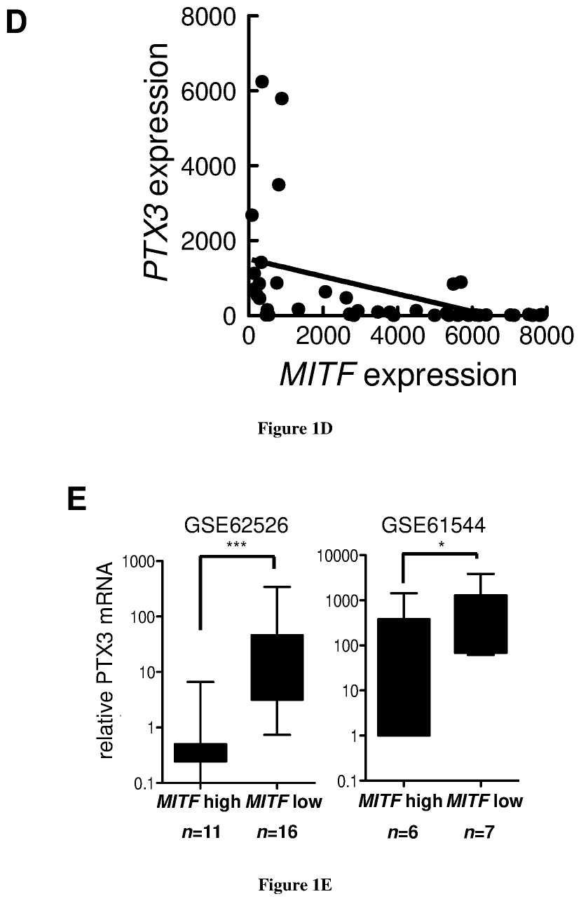

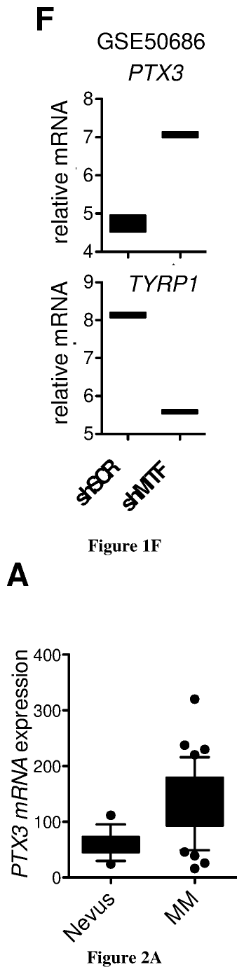

Using quantitative mass spectrometry analysis [12], the pentraxin-related protein PTX3 was found enriched in CM from metastatic 1205Lu cells compared to non-metastatic 501Mel cells (Supplementary Table 1). Antibody arrays analysis confirmed that PTX3 is abundantly produced by metastatic melanoma cells in addition to other proteins involved in migration and inflammation (IL-1α, IL-6, IL-8, MCP-1, TNFsR1, CCL2), matrix remodelling and adhesion (TSP1, TIMP1) (data not shown). Direct dosage by ELISA and immunoblot analysis of PTX3 in CM obtained from different malignant melanoma cells showed that PTX3 was preferentially produced by 1205Lu and A375 cell lines (FIG. 1a and b). PTX3 was also secreted by cultures of primary tumor cells from metastatic melanoma patients (Pt1 and Pt2) (FIG. 1a). It is recognized that melanoma cells can switch from a proliferative and poorly invasive MITFhigh phenotype to an invasive and poorly proliferative MITFlow phenotype [4]. Given our observations, we thus searched publicly available gene expression datasets for PTX3 expression in proliferative versus invasive melanoma cell population within the Mannheim, Philadelphia, and Zurich cohorts (GSE4843, GSE4841 and GSE4840 data sets, respectively) and found an inverse correlation between PTX3 and MITF expression across proliferative and invasive cell states (FIG. 1c and d). Furthermore, values extracted from additional GEO databases revealed that PTX3 expression increased in MITFlow cells compared to MITFhigh cells (GSE62526 and GSE61544) (FIG. 1e) and that shRNA-mediated depletion of MITF in COL0829 melanoma cells upregulated PTX3 mRNA expression (GSE50686) (FIG. 1f). Finally, knockdown of MITF expression in the non-metastatic cell line 501Mel was associated with increased levels and secretion of PTX3 (ldata not shown). Consistent with our previous report [5], the expression of the mesenchymal marker SPARC and p27Kip1 was also augmented following MITF depletion in 501Mel cells. Together our data identify high levels of PTX3 in MITF-low melanoma cells.

PTX3 is Upregulated During Human Metastatic Melanoma Disease and Acquisition of Mesenchymal Drug Resistance

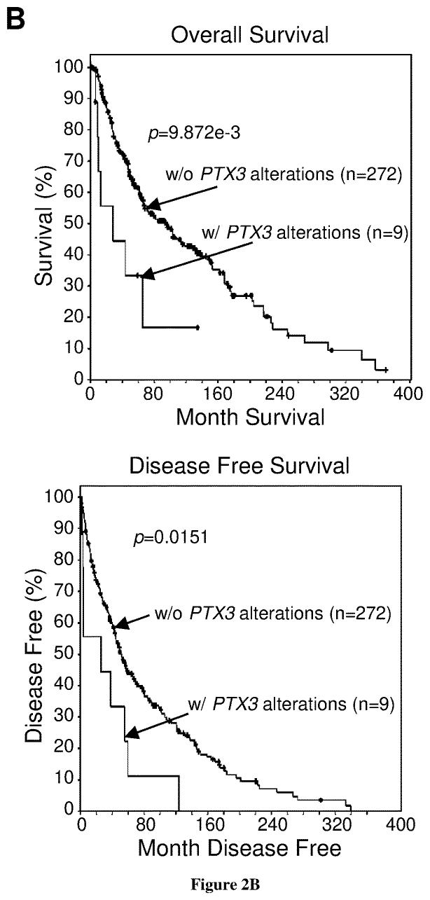

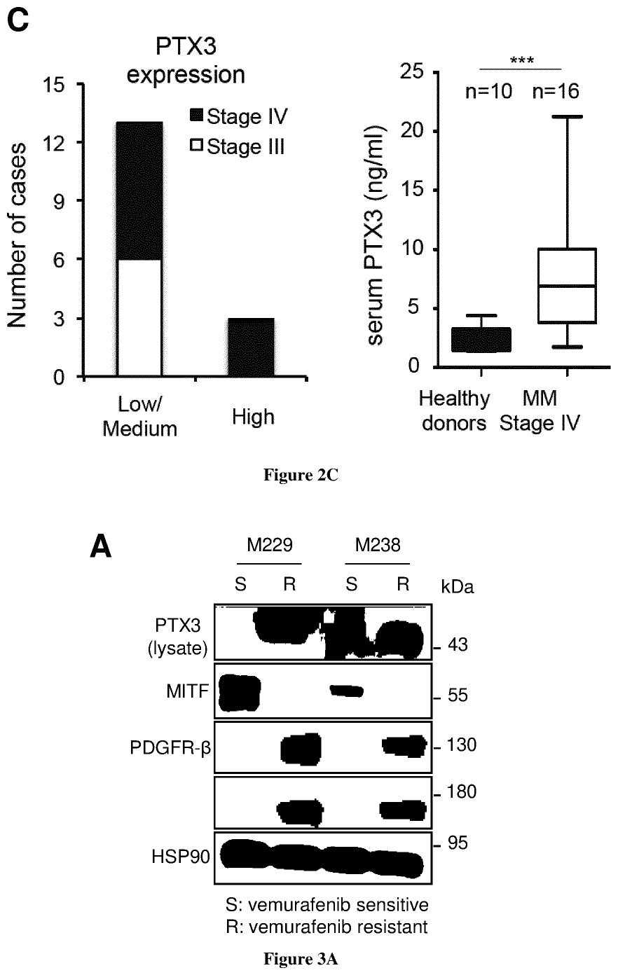

The finding that PTX3 level was associated with a melanoma invasive signature prompted us to examine its expression in metastases and blood samples from melanoma patients. In silico data analysis of PTX3 expression in public databases showed that PTX3 mRNA was increased in metastatic melanoma compared to benign skin lesions (nevi) (FIG. 2a). In addition, the examination of PTX3 in melanoma patients from The Cancer Genome Atlas (TCGA) datasets revealed that PTX3 alterations (PTX3 amplifications and mRNA upregulation) were associated with a trend toward shorter disease-free progression (p=0.0151) and shorter overall survival (p=9.872e-3) (FIG. 2b). Interestingly, gene set enrichment analysis (GSEA) showed that high PTX3 levels in melanoma tumors positively correlated with Epithelial-to-Mesenchymal Transition (EMT) and Inflammatory Response gene signatures (data not shown).

Immunohistochemical analysis of PTX3 in melanoma tissue microarrays showed that a marked PTX3 cytoplasmic and membranous immunoreactivity in malignant melanoma at stage IV (3 out of 10) compared to four adjacent normal skin tissues. In contrast, malignant melanoma at stage III (6 out of 6) showed low to medium levels of PTX3 (FIG. 2c). Consistently, the amount of circulating PTX3 in blood from patients with stage IV metastatic disease (n=16) was significantly increased in diseased (7.98±5.12 ng/ml) compared to healthy subjects (n=10) (2.36±1.15 ng/ml) samples (data not shown). These observations indicate that high PTX3 levels correlated with melanoma cells from advanced metastatic disease.

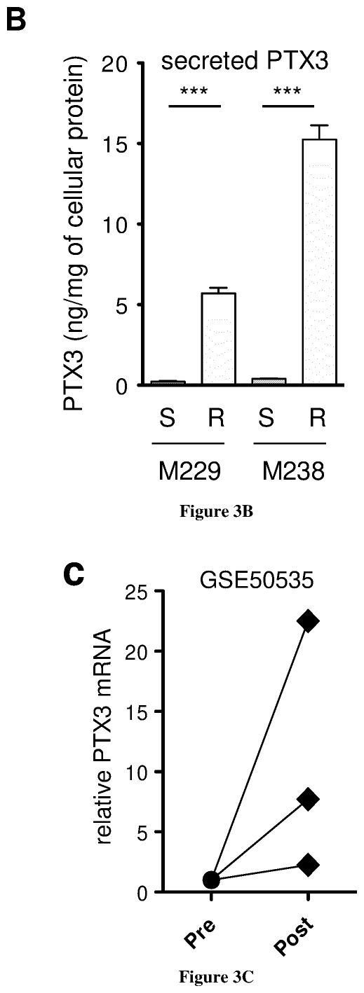

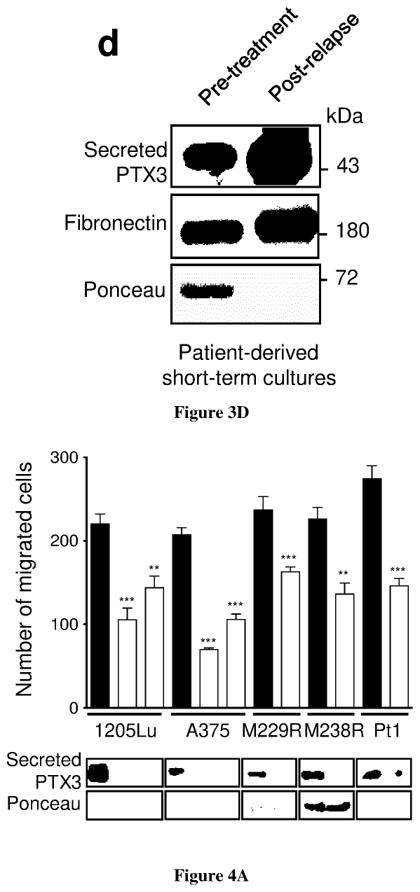

Recent findings indicate that by switching from a proliferative MITFhigh/AXLlow to a mesenchymal invasive MITFlow/AXLhigh state, melanoma cells can acquire resistance to targeted therapies [3, 7]. We thus investigated the potential link between PTX3 expression and acquired resistance to BRAF inhibitors. Immunoblot analyses performed on isogenic pairs of sensitive and vemurafenib-resistant melanoma cells [17] revealed that MITFlow resistant cells M229R and M238R expressed and secreted high levels of PTX3 when compared to MITFhigh drug-sensitive parental cells M229P and M238P (FIG. 3a and b). As expected, the mesenchymal markers Fibronectin and PDGFR-□ were also upregulated in the two resistant cell lines (FIG. 3a)[18]. Importantly, our observations were clinically supported in public gene expression datasets showing that PTX3 increased in progressing tumors biopsies during therapy with BRAFi (vemurafenib or dabrafenib) or MEKi (trametinib) (FIG. 3c). Our analysis of the CM prepared from melanoma cells derived from a relapsing melanoma patient further showed that

PTX3 and Fibronectin production by melanoma cells increased following the acquisition of mesenchymal resistance to vemurafenib (FIG. 3d). Together, our findings hence link PTX3 expression to a subpopulation of inflammatory, dedifferentiated and invasive melanoma cells, which could be associated with poor prognosis.

Melanoma-derived PTX3 Promotes Tumor Cell Migration

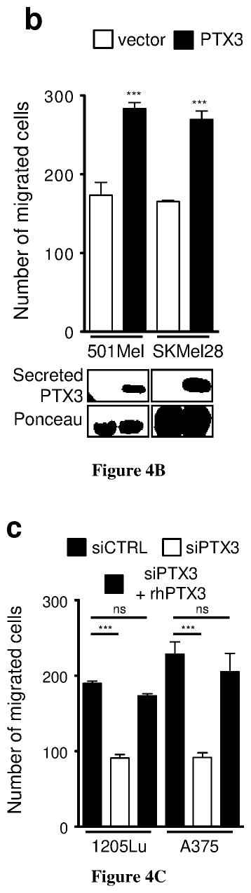

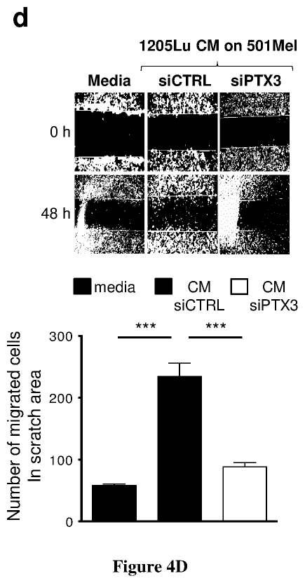

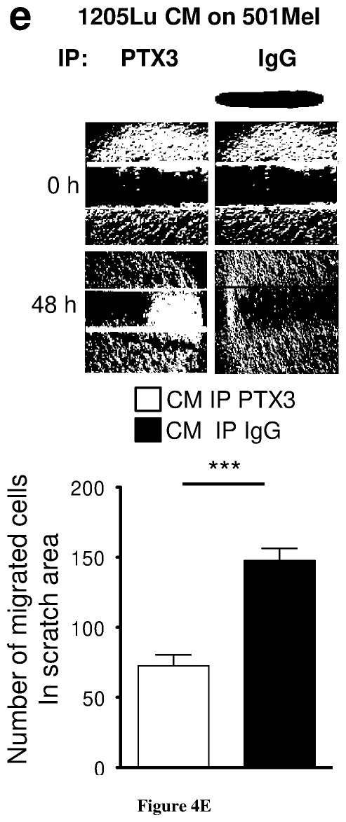

We next investigated the contribution of PTX3 to melanoma cell migration. To this end, the migratory ability of PTX3-depleted cells was tested in Boyden chamber assays. Knockdown of PTX3 in 1205Lu and A375 cells using two different non-overlapping PTX3-targeting siRNAs (siPTX3#1 and siPTX3#2) inhibited the chemotactic migration of melanoma cells towards serum compared to cells transfected with a non-targeting siRNA (siCTRL) (FIG. 4a). Importantly, PTX3 knockdown, which abrogated PTX3 secretion, had no significant effect on cell viability (data not shown). Depleting PTX3 also blocked the migration of invasive M229R and M238R BRAFi-resistant cell lines and of metastatic melanoma cells derived from a patient (Pt1) (FIG. 4a). Conversely, the stable expression of PTX3 in PTX3-negative cells 501Mel and SKMe128 increased cell migration (FIG. 4b). Addition of exogenous recombinant PTX3 rescued the migratory ability of PTX3-depleted A375 and 1205Lu cells (FIG. 4c), suggesting that PTX3 promotes melanoma cell migration in an autocrine manner following its secretion by the cancer cell. To test this hypothesis, we carried out in vitro scratch assays using the non-invasive PTX3-negative 501Mel cells incubated with CM produced by invasive PTX3-positive 1205Lu cells. As shown in FIG. 4d, the addition of CM produced by 1205Lu cells transfected by non-targeting siRNA enhanced the migration of 501Mel cells in the wound gap whereas adding CM produced by PTX3-depleted 1205Lu cells had no significant effect on 501Mel cell migration. Using 1205Lu CMs that were immuno-depleted of PTX3, we confirmed that secreted PTX3 is required to induce melanoma cell migration (FIG. 4e). Of note, PTX3-expressing 501Mel cells displayed increased cell motility in scratch assays when compared to control PTX3-negative 501Mel cells (data not shown). In addition, we found that recombinant PTX3 enhanced the migration of another non-invasive PTX3-negative melanoma cell line, SKMe128 (FIG. 4f), indicating that melanoma-derived PTX3 h as a direct effect on tumor cell motility.

PTX3 Expression Increases Melanoma Invasiveness In Vitro and In Vivo

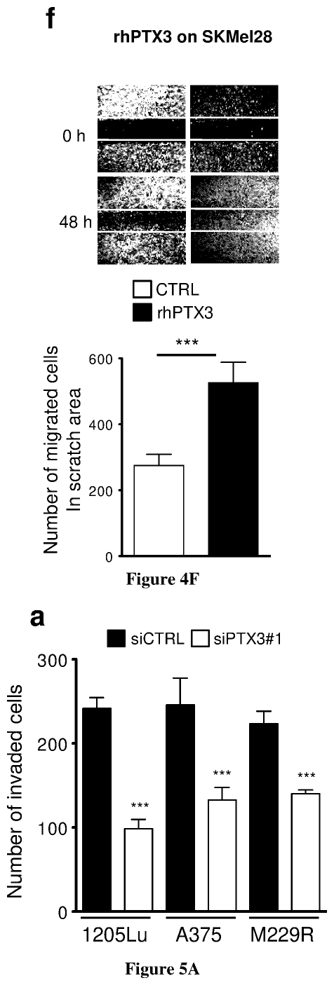

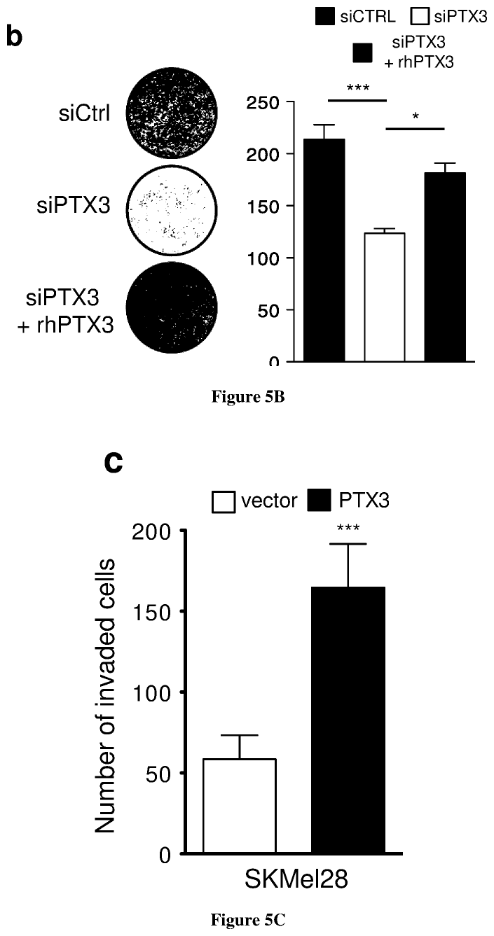

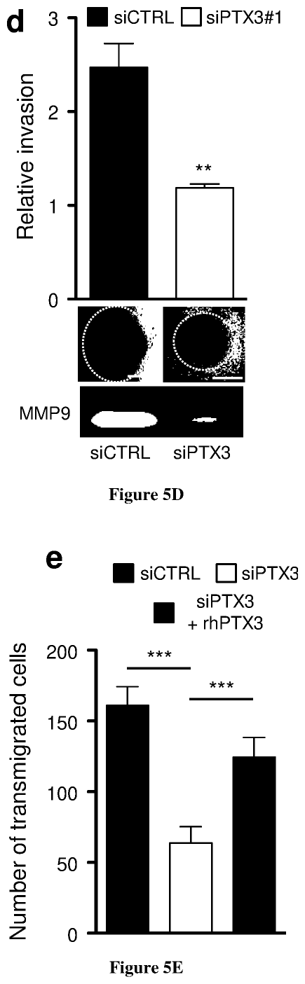

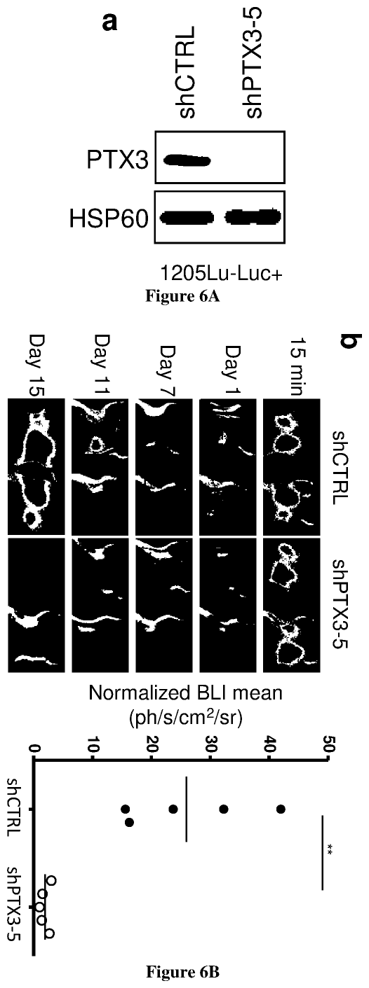

Our observations led us to investigate whether PTX3 participates to melanoma cell invasion. Matrigel invasion assays revealed that siRNA-mediated depletion of PTX3 reduced the invasive migration of 1205Lu, A375 and M229R (FIG. 5a). Interestingly, exogenous recombinant PTX3 restored the impaired invasiveness of PTX3-depleted 1205Lu cells (FIG. 5b). Conversely, stable expression of PTX3 in PTX3-negative SKMe128 cells increased the capacity of melanoma cells to invade matrigel (FIG. 5c). To investigate further the activity of PTX3 in melanoma invasion, A375 and WM266.4 cell spheroids were implanted into gels of type I collagen and 3-dimensional (3D) tumor cell invasion was evaluated by microscopy. Whereas knockdown of PTX3 h ad no significant effect on spheroid growth rate, the depletion of PTX3 impaired melanoma invasion into collagen of both cell lines (FIG. 5d). Consistently, zymographic analysis of the invasion-related matrix metalloproteinase MMP9 revealed a decrease of its collagenase activity in PTX3-depleted melanoma cells compared to control (FIG. 5d). Tumour cell extravasation is a required early step during tissue colonization and metastatic spread [19]. To examine how PTX3 participates to this process, we assessed the effect of PTX3 knockdown in transendothelial migration of 1205Lu cells on monolayers of TNFα-activated HUVECs. Compared to control siRNA-transfected cells, the transmigration of PTX3-silenced melanoma cells was markedly reduced (FIG. 5d). Importantly, the reduced potential of PTX3-depleted cells to transmigrate was rescued by the addition of exogenous PTX3, thereby linking autocrine PTX3 production by melanoma cells to their ability to cross endothelial barriers (FIG. 5e).

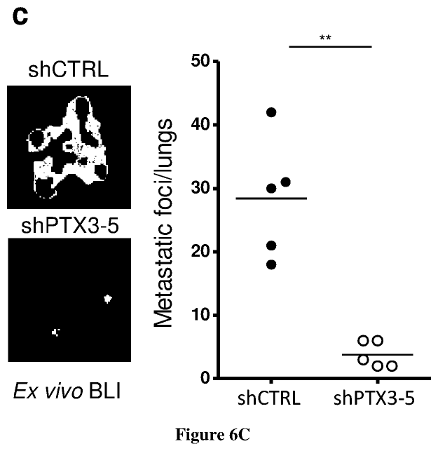

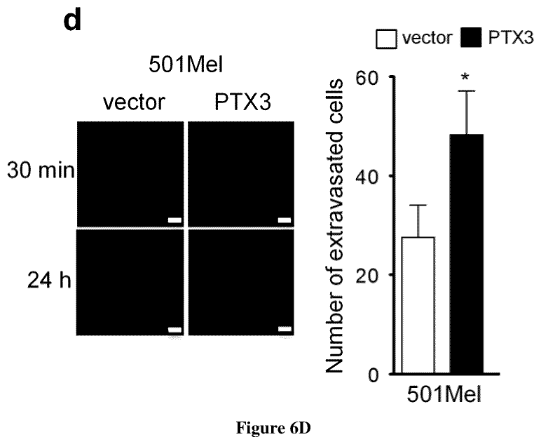

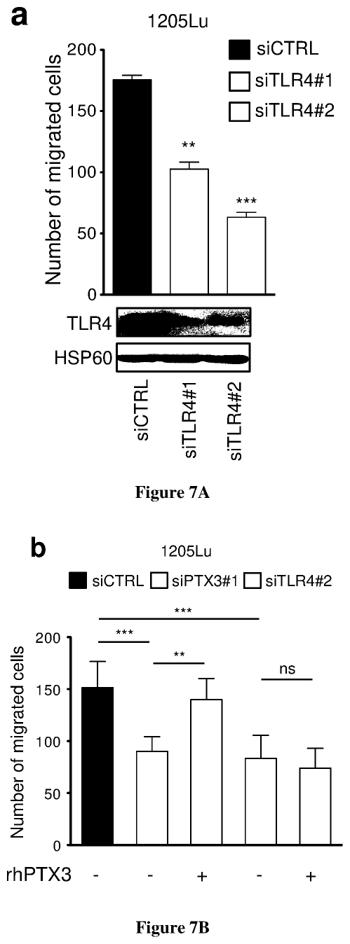

Next we sought to confirm our observations in vivo using a long-term lung colonization assay by bioluminescent melanoma cells [12]. To this end, 1205Lu-Luc+melanoma cells were stably infected with lentiviruses harbouring a control shRNA lentivirus (shCtrl) and different shRNAs against PTX3. The efficiency of PTX3 knockdown was assessed by immunoblotting conditioned media (CM) from the infected cells (data not shown). Further analysis of in vitro cell migration in Boyden chamber assays showed that shPTX3 sequence 5 (shPTX3-5) was the most efficient in reducing cell migration compared to shCtrl (data not shown). Stable control (shCTRL) and PTX3 (shPTX3-5) knockdown (FIG. 6a) bioluminescent melanoma cells were then injected into the tail vein of nude mice. Bioluminescence imaging (BLI) recorded post-injection showed that control and PTX3-depleted cells equally arrested into the lungs after 15 min. However, BLI measurements during the next 15 days revealed a dramatic reduction in lung colonization by PTX3-depleted compared to control cells (FIG. 6b). The quantification of lung colonization by ex vivo BLI at the endpoint of the assay revealed a 6-fold reduction in experimental lung metastasis by stable PTX3 knockdown cells compared to control cells (FIG. 6b). Importantly, transient PTX3 knockdown also inhibited melanoma lung extravasation in mice (data not shown). Finally, using a short-term lung colonization assay [12], we observed that expression of PTX3 in non-metastatic 501Mel cells significantly increased their extravasation into lungs (FIG. 6d). Together, these data suggest that melanoma PTX3 increased the migratory potential of melanoma cells in vivo.

A TLR4-NF-κB-TWIST1 Axis is Required for Melanoma Cell Migration Mediated by PTX3

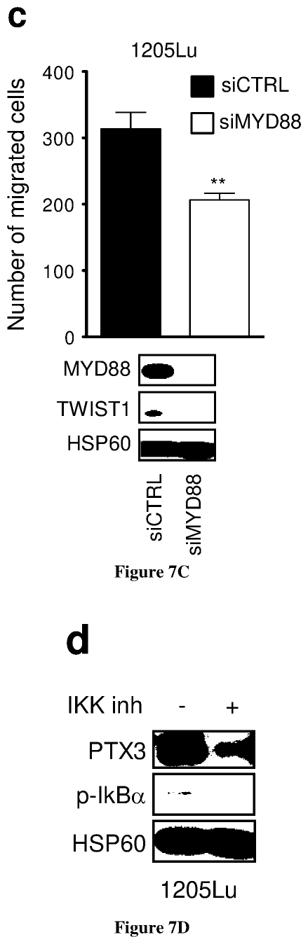

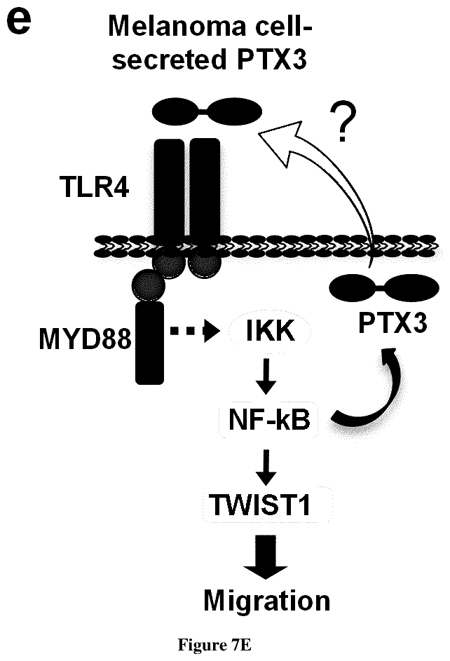

We next wished to identify the signaling pathways involved in PTX3-mediated melanoma cell migration and invasion. Whereas PTX3 depletion in 1205Lu and M229R cells had no significant effect on the activity of ERK1/2 and AKT (data not shown), it drastically decreased NF-κB pathway activity, as shown by the reduced level of phosphorylated IKKα/β, IkBα and NF-κB p65 observed in PTX3-depleted cells (data not shown). Interestingly, reduced NF-κB activity in PTX3-depleted cells decreased the expression of EMT factors TWIST1 and SNAIL. These results suggest that PTX3 activates the NF-κB pathway in melanoma to promote cell migration and invasion through EMT factors. Consistently, IKK targeting in melanoma cells either with IKK inhibitor BMS345541 (FIG. 7b) or with siRNAs directed against IKK□/□ (data not shown) reduced EMT factors levels and cell migration. In line with previous reports [10, 11, 20], we confirmed that TWIST1 is an important regulator of melanoma cell migration (data not shown). Conversely, the activation of the NF-κB pathway and TWIST1 expression was significantly increased in 501Mel cells stably expressing PTX3 compared to control cells (data not shown). Using an NF-κB-luciferase reporter construct, we confirmed that overexpression of PTX3 in melanoma cells enhanced NF-κB transcriptional activity in non-stimulated cells and showed additive effects with TNFa stimulation to further increase NF-κB activity (data not shown). Having shown that PTX3 overexpression increased the migration of poorly invasive melanoma cells, we thus examined whether the NF-κB/TWIST pathway participates to this process. A reduction of PTX3-mediated cell migration was observed in PTX3-expressing 501Mel cells treated with the IKK inhibitor BMS345541 (data not shown) or transfected with TWIST1 siRNA (data not shown). However, the migration of control cells was also reduced upon IKK inhibition or TWIST1 depletion, indicating that the NF-κB/TWIST axis controls additional PTX3-independent invasive pathways.

Finally, we sought to identify the mechanism through which melanoma-secreted PTX3 endows tumor cells with enhanced migratory potential. Our rescue experiments with exogenous recombinant PTX3 point to the existence of a PTX3 receptor that is expressed at the cell surface of melanoma cells. Previous studies showing that PTX3 mediates antifungal resistance through TLR4/MD-2-mediated signaling [21] and that melanoma cells express functional TLRs including TLR4 [22-24] led us to hypothesize that TLR4 could mediate the effect of PTX3 on melanoma cell migration. The depletion of TLR4 in melanoma cells using two siRNA sequences markedly reduced cell migration (FIG. 7a). Rescue experiments on melanoma cells that were depleted of PTX3 or TLR4 showed that recombinant PTX3 restored the migration of PTX3 silenced cells, whereas recombinant PTX3 was ineffective to restore the migration of TLR4 silenced cells (FIG. 7b). Interestingly, melanoma cells that were depleted of the TLR4 signal transducer MYD88 displayed a reduced motility, decreased NF-κB p65 phosphorylation and TWIST1 expression (FIG. 7c). Consistent with an action of PTX3 downstream TLR4/MYD88 axis, recombinant PTX3 failed to rescue migration of 1205Lu cells that were treated with a MYD88 inhibitory peptide (data not shown). Finally, we found that the IKK inhibitor BMS345541 impaired the autocrine production of PTX3 by 12051u cells (FIG. 7d). Together, our results suggest that melanoma-secreted PTX3 could act through a TLR4/MYD88 receptor complex to promote NF-κB activation, EMT factors expression and cell migration (FIG. 7e).

### REFERENCES

Throughout this application, various references describe the state of the art to which this invention pertains. The disclosures of these references are hereby incorporated by reference into the present disclosure.

1. Flaherty K T, Hodi F S, Fisher D E. From genes to drugs: targeted strategies for melanoma. Nat Rev Cancer. 2012;12(5):349-61.

2. Holderfield M, Deuker M M, McCormick F, McMahon M. Targeting RAF kinases for cancer therapy: BRAF-mutated melanoma and beyond. Nat Rev Cancer. 2014; 14(7):455-67.

3. Kemper K, de Goeje P L, Peeper D S, van Amerongen R. Phenotype switching: tumor cell plasticity as a resistance mechanism and target for therapy. Cancer Res. 2014;74(21): 5937-41.

4. Widmer D S, Cheng P F, Eichhoff O M, Belloni B C, Zipser M C, Schlegel N C, et al. Systematic classification of melanoma cells by phenotype-specific gene expression mapping. Pigment Cell Melanoma Res. 2012;25(3):343-53.

5. Cheli Y, Giuliano S, Fenouille N, Allegra M, Hofman V, Hofman P, et al. Hypoxia and MITF control metastatic behaviour in mouse and human melanoma cells. Oncogene. 2012;31(19):2461-70.

6. Verfaillie A, Imrichova H, Atak Z K, Dewaele M, Rambow F, Hulselmans G, et al. Decoding the regulatory landscape of melanoma reveals TEADS as regulators of the invasive cell state. Nature communications. 2015;6:6683.

7. Muller J, Krijgsman O, Tsoi J, Robert L, Hugo W, Song C, et al. Low MITF/AXL ratio predicts early resistance to multiple targeted drugs in melanoma. Nature communications. 2014;5:5712.

8. Shaffer S M, Dunagin M C, Torborg S R, Torre E A, Emert B, Krepler C, et al. Rare cell variability and drug-induced reprogramming as a mode of cancer drug resistance. Nature. 2017;546(7658):431-5.

9. Fenouille N, Tichet M, Dufies M, Pottier A, Mogha A, Soo J K, et al. The Epithelial-Mesenchymal Transition (EMT) Regulatory Factor SLUG (SNAI2) Is a Downstream Target of SPARC and AKT in Promoting Melanoma Cell Invasion. PLoS One. 2012;7(7):e40378.

10. Weiss M B, Abel E V, Mayberry M M, Basile K J, Berger A C, Aplin A E. TWIST1 is an ERK1/2 effector that promotes invasion and regulates MMP-1 expression in human melanoma cells. Cancer Res. 2012;72(24):6382-92.

11. Caramel J, Papadogeorgakis E, Hill L, Browne GJ, Richard G, Wierinckx A, et al. A switch in the expression of embryonic EMT-inducers drives the development of malignant melanoma. Cancer cell. 2013;24(4):466-80.

12. Tichet M, Prod'Homme V, Fenouille N, Ambrosetti D, Mallavialle A, Cerezo M, et al. Tumour-derived SPARC drives vascular permeability and extravasation through endothelial VCAM1 signalling to promote metastasis. Nature communications. 2015;6:6993.

13. Garlanda C, Jaillon S, Doni A, Bottazzi B, Mantovani A. PTX3, a humoral pattern recognition molecule at the interface between microbe and matrix recognition. Curr Opin Immunol. 2016;38:39-44.

14. Bottazzi B, Inforzato A, Messa M, Barbagallo M, Magrini E, Garlanda C, et al. The pentraxins PTX3 and SAP in innate immunity, regulation of inflammation and tissue remodelling. J Hepatol. 2016;64(6):1416-27.

15. Bonavita E, Gentile S, Rubino M, Maina V, Papait R, Kunderfranco P, et al. PTX3 is an extrinsic oncosuppressor regulating complement-dependent inflammation in cancer. Cell. 2015;160(4):700-14.

16. Magrini E, Mantovani A, Garlanda C. The Dual Complexity of PTX3 in Health and Disease: A Balancing Act? Trends Mol Med. 2016;22(6):497-510.

17. Nazarian R, Shi H, Wang Q, Kong X, Koya R C, Lee H, et al. Melanomas acquire resistance to B-RAF(V600E) inhibition by RTK or N-RAS upregulation. Nature. 2010;468(7326):973-7.

18. Titz B, Lomova A, Le A, Hugo W, Kong X, Ten Hoeve J, et al. JUN dependency in distinct early and late BRAF inhibition adaptation states of melanoma. Cell Discov. 2016;2:16028.

19. Obenauf AC, Massague J. Surviving at a Distance: Organ-Specific Metastasis. Trends Cancer. 2015;1(1):76-91.

20. Na Y R, Lee J S, Lee S J, Seok S H. Interleukin-6-induced Twist and N-cadherin enhance melanoma cell metastasis. Melanoma research. 2013;23(6):434-43.

21. Bozza S, Campo S, Arseni B, Inforzato A, Ragnar L, Bottazzi B, et al. PTX3 binds MD-2 and promotes TRIF-dependent immune protection in aspergillosis. J Immunol. 2014;193(5):2340-8.

22. Ahn J H, Park T J, Jin S H, Kang H Y. Human melanocytes express functional Toll-like receptor 4. Experimental dermatology. 2008;17(5):412-7.

23. Eiro N, Ovies C, Fernandez-Garcia B, Alvarez-Cuesta C C, Gonzalez L, Gonzalez L O, et al. Expression of TLR3, 4, 7 and 9 in cutaneous malignant melanoma: relationship with clinicopathological characteristics and prognosis. Archives of dermatological research. 2013;305(1):59-67.

24. Takazawa Y, Kiniwa Y, Ogawa E, Uchiyama A, Ashida A, Uhara H, et al. Toll-like receptor 4 signaling promotes the migration of human melanoma cells. The Tohoku journal of experimental medicine. 2014;234(1):57-65.

25. Ronca R, Di Salle E, Giacomini A, Leali D, Alessi P, Coltrini D, et al. Long pentraxin-3 inhibits epithelial-mesenchymal transition in melanoma cells. Mol Cancer Ther. 2013;12(12):2760-71.

26. Ying T H, Lee C H, Chiou H L, Yang S F, Lin C L, Hung C H, et al. Knockdown of Pentraxin 3 suppresses tumorigenicity and metastasis of human cervical cancer cells. Scientific reports. 2016;6:29385.

27. Chang W C, Wu S L, Huang W C, Hsu J Y, Chan S H, Wang J M, et al. PTX3 gene activation in EGF-induced head and neck cancer cell metastasis. Oncotarget. 2015;6(10):7741-57.

28. Chan S H, Tsai J P, Shen C J, Liao Y H, Chen B K. Oleate-induced PTX3 promotes head and neck squamous cell carcinoma metastasis through the up-regulation of vimentin. Oncotarget. 2017;8(25):41364-78.

29. Li C W, Xia W, Huo L, Lim S O, Wu Y, Hsu J L, et al. Epithelial-mesenchymal transition induced by TNF-alpha requires NF-kappaB-mediated transcriptional upregulation of Twist1. Cancer Res. 2012;72(5):1290-300.

30. Rubino M, Kunderfranco P, Basso G, Greco C M, Pasqualini F, Serio S, et al. Epigenetic regulation of the extrinsic oncosuppressor PTX3 gene in inflammation and cancer. Oncoimmunology. 2017;6(7):e1333215.

31. Choi B, Lee E J, Park Y S, Kim S M, Kim E Y, Song Y, et al. Pentraxin-3 Silencing Suppresses Gastric Cancer-related Inflammation by Inhibiting Chemotactic Migration of Macrophages. Anticancer Res. 2015;35(5):2663-8.

32. Tung J N, Ko C P, Yang S F, Cheng C W, Chen P N, Chang C Y, et al. Inhibition of pentraxin 3 in glioma cells impairs proliferation and invasion in vitro and in vivo. J Neurooncol. 2016; 129(2):201-9.

33. Thomas C, Henry W, Cuiffo B G, Collmann A Y, Marangoni E, Benhamo V, et al. Pentraxin-3 is a PI3K signaling target that promotes stem cell-like traits in basal-like breast cancers. Science signaling. 2017;10(467).

34. Giacomini A, Ghedini G C, Presta M, Ronca R. Long pentraxin 3: A novel multifaceted player in cancer. Biochim Biophys Acta Rev Cancer. 2018;1869(1):53-63.

35. Ronca R, Alessi P, Coltrini D, Di Salle E, Giacomini A, Leali D, et al. Long pentraxin-3 as an epithelial-stromal fibroblast growth factor-targeting inhibitor in prostate cancer. The Journal of pathology. 2013;230(2):228-38.

36. Ronca R, Giacomini A, Di Salle E, Coltrini D, Pagano K, Ragona L, et al. Long-Pentraxin 3 Derivative as a Small-Molecule FGF Trap for Cancer Therapy. Cancer cell. 2015;28(2):225-39.

37. Leali D, Alessi P, Coltrini D, Ronca R, Corsini M, Nardo G, et al. Long pentraxin-3 inhibits FGF8b-dependent angiogenesis and growth of steroid hormone-regulated tumors. Mol Cancer Ther. 2011;10(9):1600-10.

38. Margheri F, Serrati S, Lapucci A, Anastasia C, Giusti B, Pucci M, et al. Systemic sclerosis-endothelial cell antiangiogenic pentraxin 3 and matrix metalloprotease 12 control human breast cancer tumor vascularization and development in mice. Neoplasia. 2009;11(10):1106-15.

39. Hu F Q, Qiao T, Xie X, Hu R, Xiao H B. Knockdown of the inflammatory factor pentraxin-3 suppresses growth and invasion of lung adenocarcinoma through the AKT and NF-kappa B pathways. J Biol Regul Homeost Agents. 2014;28(4):649-57.

40. Diamandis E P, Goodglick L, Planque C, Thornquist M D. Pentraxin-3 is a novel biomarker of lung carcinoma. Clinical cancer research: an official journal of the American Association for Cancer Research. 2011;17(8):2395-9.

41. Tothill R W, Tinker A V, George J, Brown R, Fox S B, Lade S, et al. Novel molecular subtypes of serous and endometrioid ovarian cancer linked to clinical outcome. Clinical cancer research: an official journal of the American Association for Cancer Research. 2008;14(16):5198-208.

42. Kondo S, Ueno H, Hosoi H, Hashimoto J, Morizane C, Koizumi F, et al. Clinical impact of pentraxin family expression on prognosis of pancreatic carcinoma. Br J Cancer. 2013;109(3):739-46.

43. Carmo R F, Aroucha D, Vasconcelos L R, Pereira L M, Moura P, Cavalcanti M S. Genetic variation in PTX3 and plasma levels associated with hepatocellular carcinoma in patients with HCV. J Viral Hepat. 2016;23(2):116-22.

44. Locatelli M, Ferrero S, Martinelli Boneschi F, Boiocchi L, Zavanone M, Maria Gaini S, et al. The long pentraxin PTX3 as a correlate of cancer-related inflammation and prognosis of malignancy in gliomas. J Neuroimmunol. 2013;260(1-2):99-106.

45. Ravenna L, Sale P, Di Vito M, Russo A, Salvatori L, Tafani M, et al. Up-regulation of the inflammatory-reparative phenotype in human prostate carcinoma. Prostate. 2009;69(11):1245-55.

46. Coussens L M, Werb Z. Inflammation and cancer. Nature. 2002;420(6917):860-7.

47. Karin M, Greten F R. NF-kappaB: linking inflammation and immunity to cancer development and progression. Nat Rev Immunol. 2005;5(10):749-59.

48. Shao Y, Le K, Cheng H, Aplin A E. NF-kappaB Regulation of c-FLIP Promotes TNFalpha-Mediated RAF Inhibitor Resistance in Melanoma. The Journal of investigative dermatology. 2015;135(7):1839-48.

49. Riesenberg S, Groetchen A, Siddaway R, Bald T, Reinhardt J, Smorra D, et al. MITF and c-Jun antagonism interconnects melanoma dedifferentiation with pro-inflammatory cytokine responsiveness and myeloid cell recruitment. Nature communications. 2015;6:8755.

50. Didier R, Mallavialle A, Ben Jouira R, Domdom M A, Tichet M, Auberger P, et al. Targeting the proteasome-associated deubiquitinating enzyme USP14 impairs melanoma cell survival and overcomes resistance to MAPK-targeting therapies. Mol Cancer Ther. 2018;(in press).

51. Bailet O, Fenouille N, Abbe P, Robert G, Rocchi S, Gonthier N, et al. Spleen tyrosine kinase functions as a tumor suppressor in melanoma cells by inducing senescence-like growth arrest. Cancer Res. 2009;69(7):2748-56.

52. Robert G, Gaggioli C, Bailet O, Chavey C, Abbe P, Aberdam E, et al. SPARC represses E-cadherin and induces mesenchymal transition during melanoma development. Cancer Res. 2006;66(15):7516-23.

53. Fenouille N, Robert G, Tichet M, Puissant A, Dufies M, Rocchi S, et al. The p53/p21(Cip1/Waf1) pathway mediates the effects of SPARC on melanoma cell cycle progression. Pigment Cell Melanoma Res. 2011;24(1):219-32.

54. Sun C, Wang L, Huang S, Heynen GJ, Prahallad A, Robert C, et al. Reversible and adaptive resistance to BRAF(V600E) inhibition in melanoma. Nature. 2014;508(7494): 118-22.

55. Ohanna M, Cerezo M, Nottet N, Bille K, Didier R, Beranger G, et al. Pivotal role of NAMPT in the switch of melanoma cells toward an invasive and drug-resistant phenotype. Genes & development. 2018;32(5-6):448-61.

1. A method for predicting the survival time of a subject suffering from melanoma, aggressive/invasive melanoma, metastatic melanoma or melanoma resistant comprising the steps of i) quantifying the expression level of PTX3 in a biological sample obtained from the subject; ii) comparing the expression level quantified at step i) with its predetermined reference value and iii) concluding that the subject will have a short survival time when the expression level of PTX3 is higher than its predetermined reference value or concluding that the subject will have a long survival time when the expression level of PTX3 is lower than its predetermined reference value.

2. A method for treating melanoma, aggressive/invasive melanoma, metastatic melanoma or resistant melanoma in a subject in need thereof comprising a step of administering to said subject a therapeutically effective amount of an inhibitor of PTX3.

3. The method according to claim 2, wherein, the subject is identified as having a short survival time by performing the method according to claim 1.

4. The method according to claim 2, wherein, the inhibitor of PTX3 is a small organic molecule.

5. The method according to claim 2 wherein, the inhibitor of PTX3 is siRNA.

6. The method according to claim 2, wherein, the resistant melanoma is resistant to a treatment with inhibitors of BRAF mutations.

7. The method according to claim 2, wherein, the resistant melanoma is resistant to a treatment with inhibitors of MEK.

8. The method according to claim 2, wherein, the resistant melanoma is resistant to a treatment with inhibitors of NRAS.

9. The method according to claim 2, wherein, the melanoma is a double-negative BRAF and NRAS mutant melanoma.

10. The method according to claim 2, wherein, the resistant melanoma is resistant to a treatment with an immune checkpoint inhibitor.

11. The method of claim 2, further comprising providing a radiotherapy to the subject.

12. A composition comprising i) a PTX3 inhibitor and ii) an immune checkpoint inhibitor.

13. A composition comprising i) a PTX3 inhibitor and ii) a BRAF inhibitor.

14. A composition comprising i) a i) A PTX3 inhibitor and ii) a MEK inhibitor.

15. A kit for use in the method according to claim 1, said kit comprising a reagent that specifically reacts with PTX3 mRNA or protein and instructions.

16. The method of claim 2, further comprising providing an immune checkpoint inhibitor to the subject.

17. The method of claim 2, further comprising providing a BRAF inhibitor to the subject.

18. The method of claim 2, further comprising providing a MEK inhibitor to the subject.


SEQUENCE LISTING (3 sequences)

SEQ ID NO: 1 - DNA, 59 bp, Artificial
  Synthetic shRNA targerting PTX3 (shPTX3-5)
ccgggaggagctcagtatgtttcatctcgagatgaaacatactgagctcctcttttttg

SEQ ID NO: 2 - DNA, 33 bp, Artificial
  Synthetic PTX3 cDNA Fwd
actcgagccaccatgcatctccttgcgattctg

SEQ ID NO: 3 - DNA, 30 bp, Artificial
  Synthetic PTX3 cDNA Rev
taggatccttatgaaacatactgagctcct